(19) United States
(12) Patent Application Publication
(10) Pub. No.: US 2023/0383278 A1
(43) Pub. Date: Nov. 30, 2023

(54) NOVEL ADENO-ASSOCIATED VIRAL (AAV) VECTORS TO TREAT HEREDITARY METHYLMALONIC ACIDEMIA (MMA) CAUSED BY METHYLMALONYL-COA MUTASE (MMUT) DEFICIENCY

(71) Applicants: Charles P. Venditti, Potomac, MD (US); Randy Chandler, Chevy Chase, MD (US)

(72) Inventors: Charles P. Venditti, Potomac, MD (US); Randy Chandler, Chevy Chase, MD (US)

(73) Assignee: The United States of America,as represented by the Secretary,Department of Health and Human Services, Bethesda, MD (US)

(21) Appl. No.: 18/026,421

(22) PCT Filed: Sep. 16, 2021
(86) PCT No.: PCT/US2021/050699

§ 371 Date: Mar. 15, 2023

Related U.S. Application Data
(60) Provisional application No. 63/080,337, filed on Sep. 18, 2020.

(51) Int. Cl. C12N 9/90; C12N 15/86; A61K 48/00; C12Q 1/6883; A61P 3/00

(52) U.S. Cl. CPC C12N 9/90; C12N 15/86; A61K 48/005; C12Q 1/6883; A61P 3/00; C12Y 504/99002; C12Q 2600/106; C12Q 2600/158

(57) ABSTRACT

The present disclosure provides adeno-associated viral vectors, recombinant adeno-associated virus (rAAV) and methods of using such vectors and viruses in gene therapy for treating methylmalonic acidemia in patients with methylmalonyl-coA mutase (MVMUT) deficiency. Also provided are pharmaceutical compositions comprising recombinant adeno-associated virus (rAAV) and a pharmaceutically acceptable carrier or excipient.

### CROSS-REFERENCE TO RELATED APPLICATIONS

This patent application claims the benefit of co-pending U.S. Provisional Patent Application No. 63/080,337 filed Sep. 18, 2020, which is incorporated by reference in its entirety herein.

### STATEMENT REGARDING FEDERALLY SPONSORED RESEARCH AND DEVELOPMENT

This invention was made with Government support under project number ZIA HG200318-15 by the National Institutes of Health, National Human Genome Research Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: one 55,968 Byte ASCII (Text) file named “757163_ST25.txt,” created Sep. 15, 2021.

### FIELD OF THE INVENTION

The present disclosure relates generally to adeno-associated viral vectors, adeno-associated virus, and methods of their use in gene therapy for treating methylmalonic acidemia (MMA).

### BACKGROUND OF THE INVENTION

Isolated Methylmalonic Acidemia (MMA) comprises a relatively common and heterogeneous group of inborn errors of metabolism. The most common cause of isolated MMA is genetic deficiency of the enzyme methylmalonyl-coA mutase (MMUT), which, unfortunately, is also the most clinically severe. Most affected individuals display severe multisystemic disease characterized by metabolic instability, chronic renal disease, and neurological complications. The treatment of MMA entails adherence to a low protein diet, camitine supplementation and vigilant clinical monitoring. However, despite meticulous medical management, patients with MMA caused by MMUT deficiency suffer from substantial mortality and morbidity related to the disease. Most commonly, premature death occurs in the setting of an acute metabolic crisis, and has led to the use of elective liver or liver/kidney transplantation as a treatment some patients. While these experimental surgical procedures do not completely cure the biochemical phenotype of methylmalonic acidemia, metabolic stability is restored after liver transplantation, and the propensity for acute decompensation is totally eliminated. However, the patients must adhere to a life-long regimen of anti-rejection and immune suppression medicines and suffer the sequelae experienced by all transplant recipients, including the risk for graft failure, rejection and an increased incidence of malignancy.

### BRIEF SUMMARY OF THE INVENTION

The gravity of MMA, ineffectiveness of medical therapies to treat patients, and benefits experienced by liver transplant patients with respect to metabolic stability have led to the development of systemic and liver-directed gene therapy, as new treatments for MMA.

The present disclosure provides compositions and methods of their use in gene therapy. More specifically, provided herein are recombinant adeno-associated viral vectors (rAAV) comprising an adeno-associated virus (AAV) capsid, and a vector genome packaged therein useful for the treatment of MMA.

In one aspect, the present disclosure provides a recombinant adeno-associated virus (rAAV) comprising an AAV capsid, and a vector genome packaged therein, said vector genome comprising in 5′ to 3′ order: (a) a 5′-inverted terminal repeat sequence (5′-ITR) sequence; (b) a promoter sequence; (c) optionally, an intron sequence; (d) a partial or complete coding sequence for MMUT which may be codon optimized; (e) optionally, a Hepatitis B virus posttranscriptional regulatory element; (f) a polyadenylation signal which can be bovine growth hormone polyA or rabbit beta-globin polyA; and (g) a 3′-inverted terminal repeat sequence (3′-ITR) sequence.

In another aspect, the present disclosure provides a rAAV comprising an AAV capsid, and a vector genome packaged therein, said vector genome comprising in 5′ to 3′ order: (a) a 5′-inverted terminal repeat sequence (5′-ITR) sequence; (b) an enhancer sequence; (c) a promoter sequence; (d) optionally, an intron sequence; (e) a partial or complete coding sequence for MMUT which may be codon optimized; (f) optionally, a Hepatitis B virus posttranscriptional regulatory element; (g) a polyadenylation signal which can be bovine growth hormone polyA or rabbit beta-globin polyA; and (h) a 3′-inverted terminal repeat sequence (3′-ITR) sequence.

In another aspect, the present disclosure provides a recombinant adeno-associated virus (rAAV) comprising an AAV capsid, and a vector genome packaged therein, said vector genome comprising in 5′ to 3′ order: (a) a 5′-inverted terminal repeat sequence (5′-ITR) sequence; (b) a promoter sequence; (c) optionally, an intron sequence; (d) optionally, a translational enhancer element (TEE); (e) partial or complete coding sequence for MMUT which may be codon optimized; (f) optionally, a Hepatitis B virus posttranscriptional regulatory element; (g) a polyadenylation sequence which can be bovine growth hormone polyA or rabbit beta-globin polyA; and (h) a 3′-inverted terminal repeat sequence (3′-ITR) sequence.

In another aspect, the present disclosure provides a rAAV comprising an AAV capsid, and a vector genome packaged therein, said vector genome comprising in 5′ to 3′ order: (a) a 5′-inverted terminal repeat sequence (5′-ITR) sequence; (b) an enhancer sequence; (c) a promoter sequence; (d) optionally, an intron sequence; (e) optionally, a translational enhancer element (TEE); (f) a partial or complete coding sequence for MMUT which may be codon optimized; (g) optionally, a Hepatitis B virus posttranscriptional regulatory element; (h) a polyadenylation sequence which can be bovine growth hormone polyA or rabbit beta-globin polyA; and (i) a 3′-inverted terminal repeat sequence (3′-ITR) sequence.

In one embodiment, the partial or complete nucleotide coding sequence for MMUT is a wild-type coding sequence. In an alternative embodiment, the partial or complete nucleotide coding sequence for MMUT is codon-optimized. In one exemplary embodiment, the partial or complete nucleotide coding sequence for MMUT is codon-optimized for expression in humans.

In some embodiments, MMUT is encoded by the wild-type coding sequence shown in SEQ ID NO: 1. In another embodiment, the nucleotide coding sequence expressing a natural isoform or variant of the corresponding MMUT isoform may be used. In certain embodiments, MMUT is encoded by a codon-optimized nucleotide sequence. In some embodiments, MMUT is encoded by a codon-optimized nucleotide sequence that is less than about 85% identical to the wild-type coding sequence shown in SEQ ID NO: 1. In some exemplary embodiments, MMUT is encoded by a codon-optimized nucleotide sequence such as SEQ ID NO: 2. In some embodiments, the coding sequence for MMUT may further comprise a stop codon (TGA, TAA, or TAG) at the 3′ end. In some embodiments, the expressed MMUT comprises or consists of an amino acid sequence of SEQ ID NO: 3.

In some embodiments, the 5′-ITR sequence is from AAV2. In some embodiments, the 3′-ITR sequence is from AAV2. In some embodiments, the 5′-ITR sequence and the 3′-ITR sequence are from AAV2. In some embodiments, the 5′-ITR sequence and/or the 3′-ITR sequence comprise or consist of SEQ ID NO: 4. In another embodiment, the 5′-ITR sequence and/or the 3′-ITR sequence comprise or consist of SEQ ID NO: 5. In another embodiment, the 5′-ITR sequence and/or the 3′-ITR sequence comprises or consists of SEQ ID NO: 22.

In other embodiments, the 5′-ITR sequence and/or the 3′-ITR sequence are from a non-AAV2 source.

In one embodiment, the promoter is selected from an elongation factor 1 alpha promoter (EF1α), a human alpha 1-antitrypsin promoter (hAAT), chicken β-actin (CBA) promoter, a cytomegalovirus (CMV) immediate early gene promoter, a transthyretin (TTR) promoter, a thyroxine binding globulin (TBG) promoter, an alpha-1 anti-trypsin (A1AT) promoter, a CAG promoter (constructed using the CMV early enhancer element, the promoter, the first exon, and the first intron of CBA gene, and the splice acceptor of the rabbit beta-globin gene), or the endogenous MMDUT promoter. In an exemplary embodiment, the promoter is the EF1α promoter. In one embodiment, the EF1α promoter, also called EF1L, comprises or consists of SEQ ID NO: 6. In another embodiment, the EF1α promoter comprises or consists of SEQ ID NO: 7. In another exemplary embodiment, the promoter is the hAAT a promoter. In one embodiment, the hAAT promoter comprises or consists of SEQ ID NO: 8. In another embodiment, the hAAT promoter comprises or consists of SEQ ID NO: 9. In some embodiments, the promoter is a gene-specific endogenous promoter. In one some embodiment, the promoter comprises native gene promoter elements.

In some embodiments, the packaged vector genome further comprises one or more enhancer sequences. In one embodiment, the enhancer is selected from an apolipoprotein E (ApoE) enhancer, a cytomegalovirus (CMV) immediate early gene enhancer, a transthyretin enhancer (enTTR), a chicken β-actin (CBA) enhancer, or an En34 enhancer. In an exemplary embodiment, the enhancer is the ApoE enhancer. In one embodiment, the ApoE enhancer comprises or consists of SEQ ID NO: 10. In another embodiment, the ApoE enhancer comprises or consists of SEQ ID NO: 11. In certain embodiments, the enhancer is located upstream of the promoter sequence.

In some embodiments, the packaged vector genome further comprises one or more intron sequences. In one embodiment, the intron is selected from a chimeric intron, a hemoglobin subunit beta intron, an SV40 Small T intron, a rabbit hemoglobin subunit beta (rHBB) intron, a human beta globin IVS2 intron, a β-globin/IgG chimeric intron, or an hFIX intron. In one exemplary embodiment, the intron is the chimeric intron. In one embodiment, the chimeric intron sequence comprises or consists of SEQ ID NO: 12. In another exemplary embodiment, the intron is the hemoglobin subunit beta intron. In one embodiment, the hemoglobin subunit beta intron sequence comprises or consists of SEQ ID NO: 13. In yet another embodiment, the intron is synthetic and comprises or consists of SEQ ID NO: 14.

In some embodiments, the transgene cassette contains one or more translational enhancer (TEE) sequences placed between the 5′end of the transgene mRNA and the initiator codon of MMUT. In one embodiment, the TEE is selected from the 5′ untranslated region of an mRNA highly expressed in the liver, such as SERPINA 1, 3 or ALBUMIN.

In some embodiments, the packaged vector genome further comprises a polyadenylation signal sequence and a post translational response element. In a specific embodiment, the hepatitis B post translational response element comprises of, or consisting of, SEQ ID NO: 15 precedes the polyadenylation sequence.

In certain embodiments, the polyadenylation signal sequence is selected from a bovine growth hormone (BGH) polyadenylation signal sequence, a rabbit beta globin polyadenylation signal sequence, an SV40 polyadenylation signal sequence, and a MMUT gene-specific endogenous polyadenylation signal sequence. In an exemplary embodiment, the polyadenylation signal sequence is the bovine growth hormone (BGH) polyadenylation signal sequence. In one embodiment, the BGH polyadenylation signal sequence comprises or consists of SEQ ID NO: 16. In another exemplary embodiment, the polyadenylation signal sequence is the rabbit beta globin polyadenylation signal sequence. In one embodiment, the rabbit beta globin polyadenylation signal sequence comprises or consists of SEQ ID NO: 17.

In certain embodiments, the AAV capsid is from an AAV of serotype 1, 2, 3, 4, 5, 6, 7, 8, 9, 10, 11, 12, 13, rh10, rh8, 44.9, or hu37. In one exemplary embodiment, the AAV capsid is from AAV9. In another exemplary embodiment, the AAV capsid is from AAV8. In another exemplary embodiment, the AAV capsid is an AAV9 variant capsid.

In some aspects, the present disclosure provides codon-optimized nucleic acid sequences encoding MMUT. In one embodiment, the codon-optimized nucleic acid sequence encoding MMUT is less than about 80% identical to the wild-type coding sequence shown in SEQ ID NO: 1. In some embodiments, the codon-optimized nucleic acid sequence encoding MMUT is at least about 80%, about 85%, about 90%, about 91%, about 92%, about 93%, about 94%, about 95%, about 96%, about 97%, about 98%, about 99% or more identical to SEQ ID NO: 2. Further provided are fragments of the nucleic acid sequences shown in SEQ ID NO: 2 which encodes a polypeptide having functional MMUT activity. In some embodiments, the nucleic acid sequence encoding MMUT may further comprise a stop codon (TGA, TAA, or TAG) at the 3′ end.

In certain embodiments, the present disclosure provides recombinant adeno-associated virus (rAAV) useful as agents for gene therapy in the treatment of MMA, wherein said rAAV comprises an AAV capsid, and a vector genome as described herein packaged therein. In some embodiments, the AAV capsid is from an AAV of serotype 1, 2, 3, 4, 5, 6, 7, 8, 9, 10, 11, 12, 13, rh10, rh8, 44.9, or hu37 (i.e. AAV1, AAV2, AAV3, AAV4, AAV5, AAV6, AAV7, AAV9, AAV 10, AAV11, AAV12, AAV13, AAVrh10, AAVrh9, AAV 44.9 or AAVhu37). In an exemplary embodiment, the AAV vector is an AAV serotype 9 (AAV 9) vector, an AAV9 variant vector, an AAV serotype 8 (AAV8) vector, or an AAV serotype 2 (AAV2) vector.

In certain embodiments, the present disclosure provides an rAAV useful for the treatment of methylmalonic acidemia (MMA), said rAAV comprising an AAV capsid, and a vector genome packaged therein, said vector genome comprising as operably linked components in 5′ to 3′ order: (a) a 5′-ITR sequence; (b) a promoter sequence; (c) optionally, an intron sequence; (d) a coding sequence for MMUT (e) optionally, a Hepatitis B virus posttranscriptional regulatory element (HPRE); (f) a polyadenylation signal; and (g) a 3′-ITR sequence.

In certain embodiments, the present disclosure provides an rAAV useful for the treatment of MMA, said rAAV comprising an AAV capsid, and a vector genome packaged therein, said vector genome comprising as operably linked components in 5′ to 3′ order: (a) 5′-ITR sequence; (b) an enhancer sequence; (c) a promoter sequence; (d) optionally, an intron sequence; (e) a coding sequence for MMUT; (f) optionally, a HPRE; (g) a polyadenylation signal; and (h) a 3′-ITR sequence.

In certain embodiments, the present disclosure provides an rAAV useful for the treatment of MMA, said rAAV comprising an AAV capsid which is a AAV8 or AAV9, and a vector genome packaged therein, said vector genome comprising as operably linked components in 5′ to 3′ order: (a) a 5′-ITR sequence; (b) a EF1α promoter sequence; (c) optionally, an intron sequence which is either a chimeric intron or a hemoglobin subunit beta intron; (d) a coding sequence for MMUT; (e) optionally, a HPRE; (f) a polyadenylation signal which is a bovine growth hormone PolyA or rabbit beta-globin polyA; and (g) a 3′-ITR sequence.

In certain embodiments, the present disclosure provides an rAAV useful for the treatment of MMA, said rAAV comprising an AAV capsid which is a AAV8 or AAV9, and a vector genome packaged therein, said vector genome comprising as operably linked components in 5′ to 3′ order: (a) a 5′-ITR sequence; (b) an APOE enhancer element; (c) a hAAT promoter sequence; (d) optionally, an intron sequence which is either a chimeric intron or a hemoglobin subunit beta intron; (e) a coding sequence for MMUT (f) optionally, a HPRE; (g) a polyadenylation signal which is a bovine growth hormone PolyA or rabbit beta-globin polyA; and (h) a 3′-ITR sequence.

The present disclosure further relates to pharmaceutical compositions comprising an rAAV disclosed herein. In some embodiments, the pharmaceutical composition comprises a pharmaceutically acceptable carrier or excipient. In some embodiments, the pharmaceutical composition is formulated for subcutaneous, intramuscular, intradermal, intraperitoneal, or intravenous administration. In an exemplary embodiment, the pharmaceutical composition is formulated for direct administration to the liver.

In yet another aspect, the present disclosure provides methods of treating MMA in a human subject comprising administering to the human subject a therapeutically effective amount of at least one rAAV disclosed herein. In one embodiment, the present disclosure provides a method of treating MMA comprising administering an rAAV that includes an AAV capsid and a vector genome packaged therein, wherein the vector genome comprises a partial or complete coding sequence for MMUT or an isoform thereof, or a functional fragment or functional variant thereof.

In some embodiments, the rAAV is administered subcutaneously, intramuscularly, intradermally, intraperitoneally, or intravenously. In an exemplary embodiment, the rAAV is administered intravenously. In some embodiments, the rAAV is administered at a dose of about 1×10¹¹ to about 1×10¹⁴ genome copies (GC)/kg. In further embodiments, the rAAV is administered at a dose of about 1×10¹² to about 1×10¹³ genome copies (GC)/kg. In some embodiments, a single dose of rAAV is administered. In other embodiments, multiple doses of rAAV are administered.

In some aspects, provided herein are host cells comprising a recombinant nucleic acid molecule, an AAV vector, or an rAAV disclosed herein. In specific embodiments, the host cells may be suitable for the propagation of AAV. In certain embodiments, the host cell is selected from a HeLa, Cos-7, HEK293, A549, BHK, Vero, RD, HT-1080, ARPE-19, or MRC-5 cell.

The following numbered paragraphs [0032]-[0077] contain statements of broad combinations of the inventive technical features herein disclosed:
1. A synthetic methylmalonyl-CoA mutase (MMUT) polynucleotide (synMDUT) selected from the group consisting of:
a) a polynucleotide comprising the nucleic acid sequence of SEQ ID NO: 2; and
b) a polynucleotide having a nucleic acid sequence with at least 85% identity to the nucleic acid sequence of SEQ ID NO:2 and encoding a polypeptide according to SEQ ID NO:3, and having equivalent or increased expression in a host as compared to either expression of SEQ ID NO:2 or SEQ ID NO:1, wherein the polynucleotide does not have the nucleic acid sequence of SEQ ID NO:1.
2. The synthetic polynucleotide of aspect 1, wherein the polynucleotide has at least 90% identity to the nucleic acid sequence of SEQ ID NO:2.
3. The synthetic polynucleotide of aspect 1, wherein the polynucleotide has at least 95% identity to the nucleic acid sequence of SEQ ID NO:2.
4. The synthetic polynucleotide of aspect 1, wherein the polynucleotide having a nucleic acid sequence with at least 85% identity to the nucleic acid sequence of SEQ ID NO:2 and encoding a polypeptide according to SEQ ID NO:3 exhibits increased expression in the host relative to the expression SEQ ID NO:1 in the host.
5. The synthetic polynucleotide of aspect 1, wherein the synthetic polynucleotide comprises a nucleic acid sequence comprising codons that have been optimized relative to methylmalonyl-CoA mutase (MMUT) polynucleotide sequence (SEQ ID NO:1).
6. A gene expression cassette comprising the synthetic methylmalonyl-CoA mutase polynucleotide of any one of aspects 1-5.
7. The gene expression cassette of aspect 6, further comprising a 5′-inverted terminal repeat sequence (5′-ITR) sequence and a 3′-inverted terminal repeat sequence (3′-ITR) sequence.
8. The gene expression cassette of aspect 6, further comprising a promoter selected from the group consisting of chicken-beta actin promoter, the elongation factor 1 α long promoter (EF1L) (SEQ ID NO:6), the elongation factor 1 α short promoter (SEQ ID NO:7), the long variant of the human alpha 1 antitrypsin promoter (SEQ ID NO: 9) and the short variant of the human alpha 1 antitrypsin promoter (SEQ ID NO: 8).
9. The gene expression cassette of aspect 6, further comprising at least one intron selected from the group consisting of a chimeric intron (SEQ ID NO:12), modified β-globin intron (SEQ ID NO: 13), and a synthetic intron (SEQ ID NO: 14).
10. The gene expression cassette of aspect 6, further comprising an apolipoprotein E enhancer selected from the short variant of the apolipoprotein E enhancer (SEQ ID NO: 10) and the long variant of the apolipoprotein E enhancer (SEQ ID NO: 11).
11. The gene expression cassette of aspect 6, further comprising a hepatitis B response element (SEQ ID NO: 15).
12. The gene expression cassette of aspect 6, further comprising a polyadenylation sequence of bovine polyA (SEQ ID NO: 16) or rabbit polyA (SEQ ID NO: 17).
13. The gene expression cassette of aspect 6, further comprising a translation enhancer element.
14. The gene expression cassette of aspect 6, further comprising a Hepatitis B virus posttranscriptional regulatory element.
15. The gene expression cassette of aspect 6, comprised of the elements shown in FIG. 2.
16. The gene expression cassette of aspect 6, comprised of the elements shown in FIG. 3.
17. A recombinant expression vector comprising the synthetic polynucleotide of any one of aspects 1-5.
18. A recombinant expression vector comprising the gene expression cassette of any one of aspects 6-16.
19. The recombinant vector of aspect 17 or 18, wherein the vector is a recombinant adeno-associated virus (rAAV), said rAAV comprises an AAV capsid, and a vector genome packaged therein, said vector genome comprising:
(a) a 5′-inverted terminal repeat sequence (5′-ITR) sequence;
(b) a promoter sequence;
(c) partial or complete coding sequence for MMUT; and
(d) a 3′-inverted terminal repeat sequence (3′-ITR) sequence.
20. The recombinant expression vector of aspect 19, wherein the AAV capsid is from an AAV of serotype 1, 2, 3, 4, 5, 6, 7, 8, 9, 10, 11, 12, 13, rh8, 44.9, rh10, or hu37, and mutants thereof.
21. The recombinant expression vector of aspect 20, wherein the AAV capsid is from an AAV of serotype 8.
22. The recombinant expression vector of aspect 20, wherein the AAV capsid is from an AAV of serotype 9.
23. The rAAV of aspect 19, wherein the promoter is selected from the group consisting of chicken-beta actin promoter, the elongation factor 1 alpha long promoter (EF1L) (SEQ ID NO:6), the elongation factor 1 alpha short promoter (SEQ ID NO:7), the long variant of the human alpha 1 antitrypsin promoter (SEQ ID NO: 9) and the short variant of the human alpha 1 antitrypsin promoter (SEQ ID NO: 8).
24. The rAAV according to aspect 19, wherein the rAAV further comprises at least one intron selected from the group consisting of a chimeric intron (SEQ ID NO: 12), modified β-globin intron (SEQ ID NO: 13), and a synthetic intron (SEQ ID NO: 14).
25. The rAAV of aspect 19, wherein the rAAV further comprises an apolipoprotein E enhancer selected from the short variant of the apolipoprotein E enhancer (SEQ ID NO: 10) and the long variant of the apolipoprotein E enhancer (SEQ ID NO: 11).
26. The rAAV according of aspect 19, wherein the rAAV further comprises the Hepatitis B virus posttranscriptional regulatory element (SEQ ID NO: 15).
27. The rAAV of aspect 25, wherein the apolipoprotein E enhancer is selected from the group consisting of the long (SEQ ID NO: 11) and short (SEQ ID NO: 10) variant, and the human alpha 1 antitrypsin promoter, selected from the group consisting of the long (SEQ ID NO: 9) and short (SEQ ID NO: 8) variant, are operably linked and located 5′ to an intron selected from SEQ ID NO: 12-14.
28. The AAV of aspect 27, wherein the intron is a beta-globin intron (SEQ ID NO: 13).
29. The recombinant expression vector of aspect 19, wherein the promotor is a tissue specific promoter.
30. The recombinant expression vector of aspect 29, wherein the tissue specific promoter promotor is selected from the group consisting of Apo A-I, ApoE, hAAT, transthyretin, liver-enriched activator, albumin, TBG, PEPCK, and RNAP_{II} promoters (liver), PAI-1, ICAM-2 (endothelium), MCK, SMC α-actin, myosin heavy-chain, and myosin light-chain promoters (muscle), cytokeratin 18, CFTR (epithelium), GFAP, NSE, Synapsin I, Preproenkephalin, dβH, prolactin, and myelin basic protein promoters (neuronal), and ankyrin, α-spectrin, globin, HLA-DRα, CD4, glucose 6-phosphatase, and dectin-2 promoters (erythroid).
31. The recombinant expression vector of aspect 17 or 18, wherein the expression vector is AAV9 EF1L synMUT1.
32. The recombinant expression vector of aspect 17 or 18, wherein the expression vector is AAV8 EF1L synMUT1.
33. The recombinant expression vector of aspect 17 or 18, wherein the expression vector is pAAV EF1L intron C synMMUTT kana (SEQ ID NO: 18).
34. The recombinant expression vector of aspect 17 or 18, wherein the expression vector is pAAV hAAT synMMUTT Amp (SEQ ID NO: 19).
35. The recombinant expression vector of aspect 17 or 18, wherein the expression vector is pAAV9 ITR 130 EF1s intron S synMMUTT HPRE ITR 130 kana (SEQ ID NO: 20).
36. The recombinant expression vector of aspect 17 or 18, wherein the expression vector is pAAV8 ITR 130 EF1s intron S synMMUTT HPRE ITR 130 kana (SEQ ID NO: 20).
37. The recombinant expression vector of aspect 17 or 18, wherein the expression vector is pAAV9 ITR 130 EF1s intron S synMMUTT HPRE ITR 141 kana (SEQ ID NO: 21).
38. The recombinant expression vector of aspect 17 or 18, wherein the expression vector is pAAV8 ITR 130 EF1s intron S synMMUTT HPRE ITR 141 kana (SEQ ID NO: 21).
39. A composition comprising the synthetic polynucleotide of aspects 1-5 and a pharmaceutically acceptable carrier.
40. A composition comprising the recombinant expression vector of any one of aspects 17-38 and a pharmaceutically acceptable carrier.
41. A method of treating a disease or condition mediated by methylmalonyl-coA mutase, comprising administering to a subject in need thereof a therapeutic amount of a composition comprising the synthetic polynucleotide of any one of aspect 1 to 5.
42. A method of treating a disease or condition mediated by methylmalonyl-coA mutase, comprising administering to a subject in need thereof a therapeutic amount of a composition comprising the recombinant expression vector of any one of aspects 17-38.
43. A method of treating a disease or condition mediated by methylmalonyl-coA mutase, comprising administering to a subject a composition comprising a methylmalonyl-coA mutase produced using the synthetic polynucleotide of aspects 1-5.
44. The method of any one of aspects 41 to 43, wherein the disease or condition is Methylmalonic Acidemia (MMA).
45. The method of treating a disease or condition mediated by methylmalonyl-coA mutase of any one of aspects 41 to 44, comprising administering a cell isolated from a subject in need thereof the synthetic polynucleotide of aspect 1, wherein the synthetic polynucleotide is inserted into the cell of the subject via genome editing on the cell of the subject using a nuclease selected from the group of zinc finger nucleases (ZFNs), transcription activator-like effector nucleases (TALENs), the clustered regularly interspaced short palindromic repeats (CRISPER/cas system), and meganuclease re-engineered homing endonucleases on a cell from the subject; and administering the cell to the subject.
46. A method of treating a disease or condition mediated by methylmalonyl-coA mutase, comprising administering to a subject a composition comprising a methylmalonyl-coA mutase produced using the recombinant expression vector of any one of aspects 17-38.
47. The method of aspect 41, 42, 43 or 46 wherein the composition is administered subcutaneously, intramuscularly, intradermally, intraperitoneally, or intravenously.
48. The method of aspect 42, wherein the recombinant expression vector is administered at a dose of about 1×10¹¹ to about 1×10¹⁴ genome copies (GC)/kg.
49. The method of aspect 42, wherein the administering of the recombinant expression vector comprises administration of a single dose of the expression vector.
50. The method of aspect 42, wherein the administering of the recombinant expression vector comprises administration of multiple doses of the expression vector.
51. A method for monitoring treatment of a disease or condition mediated by methylmalonyl-coA mutase, wherein a therapeutic amount of a composition comprising the recombinant expression vector of any one of aspects 17-38 has been administered to a subject in need thereof, comprising detection of expression of synMMUTT using RNA in situ hybridization with a synMMUT1 nucleic acid probe.

These and other aspects and features of the invention are described in the following sections of the present disclosure.

### BRIEF DESCRIPTION OF THE DRAWINGS

The invention can be more completely understood with reference to the following drawings.

FIG. 1 is a cartoon of a representative AAV vector genome showing the hypothetical placement of the various vector elements. ITR—inverted terminal repeats; E-enhancer; P-promoter; I-intron; 5′ UTR/TEE—untranslated region/translational enhancer element; ATG-start codon; MMUT-human methymalonyl-coa mutase gene; TAA-stop codon; HPRE-Hepatitis B virus posttranscriptional regulatory element; POLYA-polyadenylation sequence; ITR-inverted terminal repeat.

FIG. 2 is an illustrative diagram showing transgenes that use an EF1α promoter to be packaged using wild-type adeno associated virus (AAV) serotype 8 or 9 capsids.

FIG. 3 is an illustrative diagram showing transgenes that use a hAAT promoter to be packaged using wild-type adeno associated virus (AAV) serotype 8 or 9 capsids.

FIG. 4 is an illustrative graph showing the results of experiments in which 3-4 adult Mmut^{-/-}; Tg^{MckMut} mice were injected with either AAV8-hAAT-synMMUT1, AAV8-CBA-synMMUT1, or AAV8-EF1L-synMMUT1. 1.5e11 genome copies (GC) of each vector were administered, roughly equating to a dose of 6e12 GC/kg, via the retro-orbital route. The plasma methylmalonic acid concentration after 10 and 30 days was measured and compared historical to untreated Mmut^{-/-}; Tg^{MckMut} controls. Each vector induced equivalent reduction in circulating metabolites.

FIG. 5 is an illustrative diagram showing the open reading frame of human methylmalonyl-coenzyme A (CoA) mutase (MMUT) encoding 742 amino acids comprising a 32 amino acid mitochondrial leader sequence and a mature protein of 710 amino acids. Murine Mmut sequence (shown) encodes a protein of 748 amino acids with a 30-amino acid leader sequence and 718-amino acid mature protein, exhibiting 94% identity with its human homologue. Patient mutations have historically been classified as either mut⁰ or mut⁻ based on MMUT activity from cultured patient fibroblasts. MMUTp.R108C (murine Mmutp.R106C), located in the region coding for the substrate binding domain, is classified as mut⁰: there is no detectable MMUT activity, resulting in the more common, and severe, form of the disorder. MMUTp.G717V (murine Mmutp.G715V) is located in the putative cofactor binding pocket and is classified as mut, exhibiting partial activity in vitro.

FIG. 6 is an illustrative graph showing the results of experiments in which only 6% of Mmut^{p.R106C/p.R106C} (n=37) survived until day of life 57, in comparison to 97% of Mmut^{+/p.R106C} (n=39) p<0.0001, log-rank Mantel-Cox and Gehan-Breslow-Wilcoxon test). The greatest mortality was observed in Mmut^{p.R106C/p.R106C} during the first one to two weeks of life, during which more than 50% of the Mmut^{p.R106C/p.R106C} mice perished. AAV9 EF1L synMMUT1 gene therapy, delivered at a dose of 1e11 GC/pup via the retroorbital plexus, rescued 100% of the treated Mmut^{p.R106C/p.R106C} mice (n=4) from neonatal lethality, and provided 75% survival past weaning and beyond. Similarly, the survival of Mmut^{p.G715V/p.G715V} (n=17) was followed, and compared to Mmut^{+/p.G715V} littermates (n=30). At 111 days, there was no significant difference in the mean survival between healthy Mmut^{+/Mutp.G715V} and diseased Mmut^{p.G715V/p.G715V} mice (log-rank Mantel-Cox and Gehan-Breslow-Wilcoxon test). Mmut^{p.G715V/p.G715V} mice did not exhibit disease-related mortality. AAV8 EF1L synMMUT1 gene therapy, indicated as AAV8 Mmut^{p.G715V/p.G715V} did not change survival.

FIG. 7 shows plasma methylmalonic acid concentrations in Mmut^{p.R106C/p.R106C} and Mmut^{p.G715V/p.G715V} mice. A litter of mice was euthanized on DOL0, with tail and liver tissue and blood samples harvested for genotyping, protein analysis and metabolite measurements, respectively. Mmut^{p.R106C/p.R106C} animals exhibited massively elevated levels of methylmalonic acid in plasma at birth (2931 μM±367, n=5; mean+/−SEM) compared to Mmut^{+/p.R106C} littermates (11.07 μM±0.7277, n=5; mean+/−SEM) [p<0.0001]. Plasma methylmalonic acid was significantly elevated in Mmut^{p.G715V/p.G715V} compared to age-matched Mmut^{+/p.G715V} controls at 16 weeks (802.5 μM±109.3, n=4 for Mmut^{p.G715V/p.G715V} versus 5.66 μM+0.7997, n=4 for Mmut^{+/p.G715V}; mean+/−SEM). Methylmalonic acid concentrations in 16-week old Mmut^{p.G715V/p.G715V} (n=3) treated with 5e12 GC/Kg AAV8 EF1L synMMUT1 dropped significantly, by over 75%, three weeks post treatment. Treated Mmut^{p.G715V/p.G715V} exhibited methylmalonic acid levels of 71.57 M+12.75, n=3, (p<0.01).

FIG. 8 is an illustrative graph showing the results of experiments in which through 4 months of life, Mmut^{p.G715V/p.G715V} display inhibited growth when fed a regular chow diet. Untreated Mmut^{p.G715V/p.G715V} only reach ˜70% of the weight of their Mmut^{+/p.G715V} littermates (p<0.05). To demonstrate that the growth impairment was a result of the pathogenic mutation, a small cohort of Mmut^{p.G715V/p.G715V} (n=3) was treated on day of life 41 with a dose of 1e13 GC/kg of adeno-associated virus serotype 8 expressing a human codon-optimized methylmalonyl-CoA mutase (AAV8 EF1L synMUT1). Treated Mmut^{p.G715V/p.G715V} increased their body weight an average of 45% within a week of treatment, and grew to be not significantly different in size from Mmut^{+/p.G715V} littermates.

FIG. 9 is an illustrative graph showing the results of experiments in which while the majority of Mmut^{p.R106C/p.R106C} mice perished by the time of weaning, (n=14) Mmut^{p.R106C/p.R106C} mice, or ˜32%, survived past weaning and were used to assess growth impairment in the Mmut^{p.R106C/p.R106C} model. Untreated Mmut^{p.R106C/p.R106C} mice were severely growth impaired compared to Mmut^{+/p.R106C} littermates at 1 month of age (7.35+/−0.79 g for n=9 Mmut^{p.R106C/p.R106C} versus 18.72+/−2.66 g for n=31 Mmut^{+/p.R106C}, p<0.0001). Treatment of Mmut^{p.R106C/p.R106C} with AAV8 EF1L synMUT1 at a dose of 5e12 GC/kg on day of life 52 improved growth to levels not significantly different from Mmut^{+/p.R106C} littermates by DOL60 (17.7 g+/−1.32 for Mmut^{p.R106C/p.R106C} (n=2); 22.5 g+/−3.30 for Mmut^{+/p.R106C} (n=31)).

FIG. 10 is an illustrative graph providing the results of experiments showing 1-¹³C-propionate oxidation capacity in Mmut^{p.G715V/p.G715V} mice. Mmut^{p.G715V/p.G715V} mice have a significantly reduced ability to oxidize propionate (n=5, closed circles) compared to their Mmut^{+/p.G715V} littermates (n=3, black squares); at 25 minutes, Mmut^{p.G715V/p.G715V} exhibit roughly 10% oxidation of propionate versus ˜37.5% in Mmut^{+/p.G715V}. 12 days after treatment with AAV8 EF1L synMMUT1 at a dose of 1e13 GC/kg, Mmut^{p.G715V/p.G715V} increased their propionate oxidation to ˜25% (n=5, triangles), to levels not significantly different from Mmut^{+/p.G715V} controls.

FIGS. 11A and 11B show two illustrative graphs providing the results of experiments showing the percentage weight change (FIG. 11A, left) and corresponding metabolic response (FIG. 11B, right) to AAV8 EF1L synMMUT1 (labeled AAV8-EF1a-synMUT1) gene therapy in Mmut^{p.G715V/p.G715V} mice was examined 12 days after systemic delivery via retroorbital injection at a dose of 5e12 VC/kg compared to 5e11 VC/kg. VC=vector copy number.

FIG. 12 is an illustrative graph providing the results of experiments showing survival after treatment with variable doses of AAV9 EF1L synMUT1 (labeled AAV9·Ef1a, MUT1.RBG). Mut^{-/-} (a.k.a. Mmut^{-/-}) mice were injected at birth and returned to their mothers, and survival was monitored. Rescue was achieved with all doses tested but was more effective with doses >1e11 GC/pup.

FIGS. 13A and 13B are illustrative graphs providing the results of experiments showing the response of adult Mmut ^{-/-}; Tgk^{MckMut} mice (n=5) to a 5e12 GC/kg dose of AAV9 EF1L synMUT1 (labeled AAV9-EF1a-MUT1-RBG) delivered by tail vein (TV) injection. Metabolites (methylmalonic acid levels in the plasma—FIG. 13A, left) and clinical response (weight gain—FIG. 13B, right) were monitored before the injection and 12 days after treatment. A robust response was noted.

FIG. 14 is an illustrative graph providing the results of experiments showing response of adult Mmut^{p.G715V/p.G715V} mice (n=8) to a high protein (HP) challenge. Mice were maintained on a regular chow diet and switched to 70% by weight casein chow (HP). Within 5 days, only the Mmut^{p.G715V/p.G715V} animals lost >20% of their body weight, were obtunded, and needed to be euthanized. The heterozygous littermates thrived and gained weight on the same diet.

FIGS. 15A-15B show two illustrative graphs providing the results of experiments showing therapeutic and protective effects of AAV9 EF1L synMMUT1 gene therapy. Adult Mmut^{p.G715V/p.G715V} mice (n=6) were administered AAV9 EF1L synMMUT1 at a dose of 5e12 GC/kg and after two weeks, were exposed to a high protein diet (70% by weight casein chow-labeled blue chow). While similarly stressed Mmut^{p.G715V/p.G715V} mice perish within a week of exposure to a high protein challenge as shown in FIG. 12, Mmut^{p.G715V/p.G715V} mice treated with AAV9 EF1L synMMUT1 tolerate a high protein challenge as proven by extended survival, but do manifest slowed growth (FIG. 15A) and increased metabolites (FIG. 15B). After resumption of a regular diet, the AAV9 EF1L synMMUT1 treated Mmut^{p.G715V/p.G715V} mice rapidly recover growth to the level of littermate controls. (15A) Baseline Pre-GT=untreated Mmut^{+/p.G715V} and Mmut^{p.G715V/p.G715V}; mice post GT pre Blue Chow=Mmut^{+/p.G715V} and Mmut^{p.G715V/p.G715V} that received AAV9 EF1L synMMUT1 at a dose of 5e12 GC/kg; 1 Week Blue Chow=Mmut^{+/p.G715V} and Mmut^{p.G715V/p.G715V} that received AAV9 EF1L synMMUT1 at a dose of 5e12 GC/kg and then a high protein diet for 1 week; 5 Weeks Blue Chow=Mmut^{+/p.G715V} and Mmut^{p.G715V/p.G715V} that received AAV9 EF1L synMMUT1 at a dose of 5e12 GC/kg and then a high protein diet for 5 weeks; 2 Weeks Regular Chow=Mmut^{+/p.G715V} and Mmut^{p.G715V/p.G715V} that received AAV9 EF1L synMMUT1 at a dose of 5e12 GC/kg and then a high protein diet for 5 weeks and then stopped the high protein diet and were fed regular chow for 2 weeks (15B) Baseline pre-GT=Baseline Pre-GT=untreated Mmut^{p.G715V/p.G715V}; post GT Pre Blue Chow=Mmut^{p.G715V/p.G715V} that received AAV9 EF1L synMMUT1 at a dose of 5e12 GC/kg and were then bled 2 weeks after injection while on a regular chow diet; 1 Week Blue Chow=Mmut^{p.G715V/p.G715V} that received AAV9 EF1L synMMUT1 at a dose of 5e12 GC/kg and then a high protein diet for 1 week.

FIGS. 16A-16B show the pattern of MMUT mRNA expression in a representative Mmut^{p.R106C/p.R106C} mouse treated at birth with AAV9 EF1L synMMUT1 gene therapy. FIG. 16A shows the expression of the synMMUT1 mRNA at 6 months in a Mmut^{p.R106C/p.R106C} mouse at lower magnification. FIG. 16B illustrates a higher magnification of a section of the same liver slice. The detection of individual cells (circled) that express synMMUT1 over the background of endogenous Mmut, with concomitant visualization of the hepatic cytoarchitecture is shown.

### DETAILED DESCRIPTION OF THE INVENTION

This invention provides a range of novel agents and compositions to be used for therapeutic applications. The nucleic acid sequences, vectors, recombinant viruses, and associated compositions of this invention can be used for ameliorating, preventing, or treating MMA as described herein.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 13 recognized serotypes of AAV (AAV1-13).

Administration/Administer: To provide or give a subject an agent, such as a therapeutic agent (e.g., a recombinant AAV), by any effective route. Exemplary routes of administration include, but are not limited to, injection (such as subcutaneous, intramuscular, intradermal, intraperitoneal, and intravenous), oral, intraductal, sublingual, rectal, transdermal, intranasal, vaginal and inhalation routes.

Codon-optimized: A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

An “effective amount” or “an amount effective to treat” or “therapeutically effective amount” refers to a dose that is adequate to prevent or treat MMA in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect.

Enhancer: A nucleic acid sequence that increases the rate of transcription by increasing the activity of a promoter.

Intron: A stretch of DNA within a gene that does not contain coding information for a protein. Introns are removed before translation of a messenger RNA.

Inverted terminal repeat (ITR): Symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV integrating vectors.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carrier: The pharmaceutically acceptable carriers (vehicles) useful in this disclosure are conventional. Remington's Pharmaceutical Sciences, by E. W. Martin, Mack Publishing Co., Easton, Pa., 22nd Edition (2012), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease (such as MMA) refers to inhibiting the full development of a disease and/or preventing recurrence of a disease. “Treating” refers to a therapeutic intervention that ameliorates a sign or symptom of a disease or pathological condition (such as MMA) after it has begun to develop. “Ameliorating” refers to the reduction in the number or severity of signs or symptoms of a disease (such as MMA).

Promoter: A region of DNA that directs/initiates transcription of a nucleic acid (e.g., a gene). A promoter includes necessary nucleic acid sequences near the start site of transcription. Many promoter sequences are known to the person skilled in the art and even a combination of different promoter sequences in artificial nucleic acid molecules is possible. As used herein, gene-specific endogenous promoter refers to native promoter element that regulates expression of the endogenous gene of interest. In an exemplary embodiment, a MMUT gene-specific endogenous promoter regulates expression of a MMUT gene.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

Similarly, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein or virus. As used herein, “recombinant AAV” refers to an AAV particle in which a recombinant nucleic acid molecule such as a recombinant nucleic acid molecule encoding MMUT has been packaged.

Sequence identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of MMA in a mammal. Furthermore, the treatment or prevention provided by the inventive method can include treatment or prevention of one or more conditions or symptoms of MMA being treated or prevented. Also, for purposes herein, “prevention” can encompass delaying the onset of the disease, or a symptom or condition thereof.

The subject can be any mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Camivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970: Pearson & Lipman, Proc. Natl. Acad. Sci. USA 85:2444, 1988; Higgins & Sharp, Gene, 73:237-44, 1988; Higgins & Sharp, CABIOS5 A51-3, 1989; Corpet et al. Nuc. Acids Res. 16: 10881-90, 1988; Huang et al. Computer Appls. in the Biosciences 8, 155-65, 1992: and Pearson et al., Meth. Mol. Rio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

Serotype: A group of closely related microorganisms (such as viruses) distinguished by a characteristic set of antigens.

Stuffer sequence: Refers to a sequence of nucleotides contained within a larger nucleic acid molecule (such as a vector) that is typically used to create desired spacing between two nucleic acid features (such as between a promoter and a coding sequence), or to extend a nucleic acid molecule so that it is of a desired length. Stuffer sequences do not contain protein coding information and can be of unknown/synthetic origin and/or unrelated to other nucleic acid sequences within a larger nucleic acid molecule.

Subject: Living multi-cellular vertebrate organisms, a category that includes human and non-human mammals.

Synthetic: Produced by artificial means in a laboratory, for example a synthetic nucleic acid can be chemically synthesized in a laboratory.

Untranslated region (UTR): A typical mRNA contains a 5′ untranslated region (“5′ UTR”) and a 3′ untranslated region (3′ UTR) upstream and downstream, respectively, of the coding region (see Mignone F. et. al., (2002) Genome Biol 3:REVIEWS0004).

Therapeutically effective amount: A quantity of a specified pharmaceutical or therapeutic agent (e.g., a recombinant AAV) sufficient to achieve a desired effect in a subject, or in a cell, being treated with the agent. The effective amount of the agent will be dependent on several factors, including, but not limited to the subject or cells being treated, and the manner of administration of the therapeutic composition.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an AAV vector.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Table 1 below provides abbreviations used in the present application

TABLE 1

| Abbreviation | Long-form meaning |
|---|---|
| MMA | methylmalonic acidemia |
| MMUT or MUT (interchangeable) | methylmalonyl-coA mutase |
| MMUT or MUT | Gene encoding |
| (italicized - interchangeable) | methylmalonyl-coA mutase |
| AAV | adeno-associated virus |
| rAAV | recombinant adeno-associated virus |
| ITR | inverted terminal repeat |
| TEE | translational enhancer element |
| HPRE | Hepatitis B virus posttranscriptional |
| | regulatory element |
| EF1α | elongation factor 1 alpha promoter |
| EF1L | elongation factor 1 alpha promoter, |
| | long |
| hAAT | human alpha 1-antitrypsin promoter |
| CBA | chicken β-actin |
| CMV | cytomegalovirus immediate |
| | early gene promoter |
| TTR | Transthyretin promoter |
| TBG | thyroxine binding globulin promoter |
| A1AT | alpha-1 anti-trypsin promoter |
| CAG | CMV early enhancer element |
| | promoter construct |
| ApoE | apolipoprotein E enhancer |
| CMV | cytomegalovirus (CMV) immediate |
| | early gene enhancer |
| enTTR | transthyretin enhancer |
| rHBB | rabbit hemoglobin subunit beta intron |
| BGH | Bovine growth hormone |
| RBG | Rabbit beta-globin poly |
| | adenylation sequence |

Viral Vectors: In some aspects, the present disclosure provides adeno-associated virus (AAV) vector that contains a packaged genome that comprises an AAV 5′-ITR, a promoter sequence, a partial or complete coding sequence for MMUT or an isoform thereof, or a functional fragment or functional variant thereof, and an AAV 3′-ITR.

In certain embodiments, the present disclosure provides an AAV vector that contains a packaged genome that comprises a 5′-ITR; a promoter sequence; optionally, an intron sequence; a partial or complete coding sequence for MMUT which may be codon optimized; optionally, a HPRE; a polyadenylation signal which can be bovine growth hormone polyA or rabbit beta-globin polyA; and a 3′-ITR sequence.

In certain embodiments, the present disclosure provides an AAV vector that contains a packaged genome that comprises a 5′-ITR; an enhancer sequence; a promoter sequence; optionally, an intron sequence; a partial or complete coding sequence for MMUT which may be codon optimized; optionally, a HPRE; a polyadenylation signal which can be bovine growth hormone polyA or rabbit beta-globin polyA; and a 3′-ITR sequence.

In some embodiments, the packaged genome may further comprise an enhancer, an intron, a consensus Kozak sequence, and/or a polyadenylation signal as described herein. In some embodiments, the recombinant vector can further include one or more staffer nucleic acid sequences. In one embodiment, a stuffer nucleic acid sequence is situated between the intron and the partial or complete coding sequence for MMUT.

In a preferred embodiment, the AAV vector transgene is completely devoid of AAV sequences other than the ITRs.

In an another preferred embodiment, the AAV vector transgene plasmid encodes a kanamycin resistance gene.

In various embodiments described herein, the recombinant virus vector is an AAV vector. The AAV vector can be an AAV vector of serotype 1, 2, 3, 4, 5, 6, 7, 8, 9, 10, 11 or 12 (i.e., AAV1, AAV2, AAV3, AAV4, AAV5, AAV6, AAV7, AAV8, AAV9, AAV 10, AAV 11 or AAV 12), as well as any one of the more than 100 variants isolated from human and nonhuman primate tissues. See, e.g., Choi et al., 2005, Curr Gene Ther. 5: 299-310, 2005 and Gao et al, 2005, Curr Gene Ther. 5: 285-297. AAV vectors of any serotype may be used in the present invention, and the selection of AAV serotype will depend in part on the cell type(s) that are targeted for gene therapy. For treatment of MMA, the liver is one of the relevant target organs. In some embodiments, the AAV vector is selected from serotype 9 (AAV9), serotype 8 (AAV8), or variant thereof. In an exemplary embodiment, the AAV vector is serotype 9 (AAV9) or a variant thereof.

In some embodiments, the recombinant AAV vector includes an AAV ITR sequence, which functions as both the origin of vector DNA replication and the packaging signal of the vector genome, when AAV and adenovirus helper functions are provided in trans. Additionally, the ITRs serve as the target for single-stranded endonucleatic nicking by the large Rep proteins, resolving individual genomes from replication intermediates.

In some embodiments, the 5′-ITR sequence is from AAV2. In some embodiments, the 3′-ITR sequence is from AAV2. In some embodiments, the 5′-ITR sequence and the 3′-ITR sequence are from AAV2. In other embodiments, the 5′-ITR sequence and/or the 3′-ITR sequence are from a non-AAV2 source.

Promoter: In various aspects described herein, viral vectors are provided, which contain a packaged genome which comprises a promoter sequence which helps drive and regulate transgene expression, e.g., expression of MMUT. In certain embodiments, the promoter is selected from an elongation factor 1 alpha (EF1a) promoter, a human alpha 1-antitrypsin (hAAT) promoter, a chicken b-actin (CBA) promoter, a cytomegalovirus (CMV) immediate early gene promoter, a transthyretin (TTR) promoter, a thyroxine binding globulin (TBG) promoter, an alpha-1 anti-trypsin (A1AT) promoter, a CAG promoter, and a MMUT gene-specific endogenous promoter. In exemplary embodiments, the promoter sequence is located between the selected 5′-ITR sequence and the partial or complete coding sequence for MMUT. In some embodiments, the promoter sequence is located downstream of an enhancer sequence. In some embodiments, the promoter sequence is located upstream of an intron sequence. In some embodiments, the promoter sequence is located between the selected 5′-ITR sequence and the truncated or complete nucleotide sequence of human MMUT 5′-untranslated region (UTR).

In addition to a promoter, a packaged genome may contain other appropriate transcription initiation, termination, enhancer sequence, and efficient RNA processing signals. As described in further detail below, such sequences include splicing and polyadenylation (poly A) signals, regulatory elements that enhance expression (i.e. WPRE), sequences that stabilize cytoplasmic mRNA, sequences that enhance translation efficiency (i.e. the Kozak consensus sequence), and sequences that enhance protein stability.

In some embodiments, the packaged genome further comprises a consensus Kozak sequence. In some embodiments, the consensus Kozak sequence is located downstream of an intron sequence. As will be understood by those skilled in the art, the consensus Kozak sequence is typically located immediately upstream of a coding sequence; in this case, immediately upstream of a partial or complete coding sequence for MMUT. As will be appreciated by the skilled artisan, the consensus Kozak sequence can be considered to share an ATG residue corresponding to the start codon of the therapeutic polypeptide, e.g., MMUT. For the simplicity of disclosure, the consensus Kozak sequence, as described herein, comprises a six-nucleotide sequence corresponding to the region not shared with the therapeutic polypeptide, e.g., MMUT.

Untranslated Region: 5′-untranslated region (UTR) from endogenous gene-specific mRNA have been known to play an important role in optimizing transgene production by competing with cellular transcripts for translation initiation factors and ribosomes, increasing mRNA half-life by minimizing mRNA decay or post-transcriptional gene silencing, and avoiding deleterious interactions with regulatory proteins or inhibitory RNA secondary structures (see Chiba, Y., and Green, P. (2009). J. Plant Biol. 52, 114-124, Moore, M. J., and Proudfoot, N. J. (2009). Cell 136, 688-700, Jackson, R. J., et. al. (2010). Nat. Rev. Mol. Cell Biol. 11, 113-127). The 3′-untranslated region (3′-UTR), situated downstream of the protein coding sequence, has been found to be involved in numerous regulatory processes including transcript cleavage, stability and polyadenylation, translation and mRNA localisation. They are thus critical in determining the fate of an mRNA (see Barrett, U. W., et. al. (2012). Cell Mol Life Sci. November; 69(21): 3613-3634). In certain embodiments, the present disclosure provides an rAAV that contains a packaged genome that comprises a truncated or complete nucleotide sequence of human MMUT 5′-UTR. In certain embodiments, the present disclosure provides an rAAV that contains a packaged genome that comprises a truncated or complete nucleotide sequence of human MMUT 3′-UTR.

In one embodiment, the partial or complete coding sequence for MMUT is a wild-type coding sequence. As used herein, the term “wild-type” refers to a biopolymer (e.g., a polypeptide sequence or polynucleotide sequence) that is the same as the biopolymer (e.g., polypeptide sequence or polynucleotide sequence) that exists in nature.

In an alternative embodiment, the partial or complete coding sequence for MMUT is a codon-optimized coding sequence. In one embodiment, the partial or complete coding sequence for MMUT is codon-optimized for expression in humans.

In various embodiments described herein, vectors are provided that contain a packaged genome that comprise a coding sequence for MMUT. The polypeptides delivered with the vectors described herein encompass MMUT polypeptides that may be useful in the treatment of mammals, including humans.

In some embodiments, the polypeptide expressed with a vector described herein is MMUT (SEQ ID NO: 3; 750 amino acids) or a functional fragment, functional variant, or functional isoform thereof. In some embodiments, the polypeptide expressed with a vector described herein is MMUT and comprises or consists of SEQ ID NO: 3.

In various aspects, the invention may be used to deliver fragments, variants, isoforms, or fusions of the MMUT polypeptides described herein.

In some embodiments, the invention may be used to deliver variants of the MMUT polypeptide. In some embodiments, the variant polypeptides may be at least about 80% (e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.5%, about 99.6%, about 99.7%, about 99.8%, about 99.9%, or about 100%) identical to the wild-type therapeutic polypeptide, e.g., a wild-type MMUT polypeptide of SEQ ID NO: 3. In some embodiments, the variant therapeutic polypeptides may have at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 different residues as compared to the respective wild-type polypeptide. Such variants may be obtained by recombinant techniques that are routine and well-known in the art. Moreover, such variants may be tested for catalytic activity by routine in vitro assays known to the skilled artisan. The invention further includes nucleic acid molecules which encode the above described therapeutic polypeptide variants.

Novel Codon-Optimized Sequences: In some aspects, the present disclosure provides novel codon-optimized nucleic acid sequences encoding MMUT. In one embodiment, the codon-optimized nucleic acid sequence encoding MMUT is less than about 80% identical to the wild-type coding sequence shown in SEQ ID NO: 1. In some embodiments, the codon-optimized nucleic acid sequence encoding MMDUT is at least about 80% (e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.5%, about 99.6%, about 99.7%, about 99.8%, about 99.9%, or about 100%) identical to SEQ ID NO: 2. In some embodiments, the codon-optimized nucleic acid sequence encoding MMDUT is 100% identical to a sequence selected from SEQ ID NO: 2. In some embodiments, the present disclosure provides nucleic acid sequences which are less than about 80% identical to the wild-type coding sequence shown in SEQ ID NO: 1 and are at least about 80%, about 85%, about 90%, about 91%, about 92%, about 93%, about 94%, about 95%, about 96%, about 97%, about 98%, about 99% or more identical to SEQ ID NO: 1. Further provided are fragments of the nucleic acid sequences shown in SEQ ID NO: 2 which encode a polypeptide having functional MMUT activity. In some embodiments, the nucleic acid sequence encoding MMUT may further comprise a stop codon (TGA, TAA, or TAG) at the 3′ end.

In some embodiments, the rAAV contains a packaged vector genome that further comprises one or more enhancer sequences. In one embodiment, the enhancer is selected from a human apolipoprotein E gene enhancer element (APOE), cytomegalovirus immediate early gene (CMV) enhancer, a transthyretin enhancer (enTTR), a chicken β-actin (CBA) enhancer, and an En34 enhancer. In an exemplary embodiment, the enhancer is the APOE enhancer. In one embodiment, the APOE enhancer comprises or consists of a sequence selected from the group consisting of SEQ ID NOs: 10 and 11.

In some embodiments, the rAAV contains a packaged vector genome that further comprises one or more intron sequences. In one embodiment, the intron is selected from a chimeric intron, a hemoglobin subunit beta intron, SV40 Small T intron, a rabbit hemoglobin subunit beta (rHBB) intron, a human beta globin IVS2 intron, a chimeric intron, or an hFIX intron. In one exemplary embodiment, the intron is the chimeric intron.

In some embodiments, the rAAV contains a packaged vector genome that further comprises a polyadenylation signal sequence. In one embodiment, the polyadenylation signal sequence is selected from a bovine growth hormone (BGH) polyadenylation signal sequence, a rabbit beta-globin polyadenylation signal sequence, an SV40 polyadenylation signal sequence, and a MMUT gene-specific endogenous polyadenylation signal sequence. In an exemplary embodiment, the polyadenylation signal sequence is the bovine growth hormone (BGH) polyadenylation signal sequence. In one embodiment, the BGH polyadenylation signal sequence comprises or consists of SEQ ID NO: 16. In another exemplary embodiment, the polyadenylation signal sequence is the rabbit beta-globin polyadenylation signal sequence. In one embodiment, the rabbit beta-globin polyadenylation signal sequence comprises or consists of SEQ ID NO: 17. In one embodiment, the polyadenylation signal sequence is a MMUT gene-specific endogeneous polyadenylation sequence. In an exemplary embodiment, the polyadenylation signal sequence is a MMUT gene-specific endogenous polyadenylation signal sequence when the partial or complete coding sequence in the vector genome is for MMUT.

In another aspect, the present disclosure provides rAAV useful as agents for gene therapy in the treatment of MMA, wherein said rAAV comprises an AAV capsid, and a vector genome as described herein packaged therein. In some embodiments, the AAV capsid is from an AAV of serotype 1, 2, 3, 4, 5, 6, 7, 8, 9, 10, 11, 12, rh10, or hu37 (i.e., AAV1, AAV2, AAV3, AAV4, AAV5, AAV6, AAV7, AAV9, AAV 10, AAV11, AAV 12, AAVrh10, or AAVhu37). In an exemplary embodiment, the AAV vector is an AAV serotype 9 (AAV9) vector, an AAV9 variant vector, an AAV serotype 8 (AAV8) vector, or an AAV serotype 2 (AAV2) vector.

The AAV9 capsid is a self-assembled AAV capsid composed of multiple AAV9 vp proteins. The AAV9 vp proteins are typically expressed as alternative splice variants encoded by a nucleic acid and protein sequences that are well known to practioners of the art. See also U.S. Pat. No. 7,906,111, and WO/2005/033321. As used herein, an AAV9 variant includes those described in, e.g., WO/2016/049230, U.S. Pat. No. 8,927,514, US Patent Publication No. 2015/0344911, and U.S. Pat. No. 8,734,809.

As indicated herein, the AAV9 sequences and proteins are useful in the production of rAAV. However, in other embodiments, another AAV capsid is selected. Tissue specificity is determined by the capsid type. AAV serotypes which transduce a suitable target (e.g., liver, muscle, lung, or CNS) may be selected as sources for capsids of AAV viral vectors including, e.g, AAV1, AAV2, AAV3, AAV3b, AAV4, AAV5, AAV6, AAV6.2, AAV7, AAV8, AAV9, AAVrh10, AAVrh64R1, AAVrh64R2, AAVrh8, AAV 13, and AAV 44.9. See, e.g., US Patent Publication No. 2007/0036760; US Patent Publication No. 2009/0197338; and EP 1310571. See also WO 2003/042397 (AAV7 and other simian AAV), U.S. Pat. Nos. 7,282,199 and 7,790,449 (AAV8). In addition, AAV yet to be discovered, or a recombinant AAV based thereon, may be used as a source for the AAV capsid. These documents also describe other AAV which may be selected for generating AAV and are incorporated by reference. In some embodiments, an AAV capsid for use in the viral vector can be generated by mutagenesis (e.g. by insertions, deletions, or substitutions) of one of the aforementioned AAV capsids or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned capsids.

A suite of mouse models that allow testing of new therapies for MMA caused by MMUT deficiency have been disclosed in Manoli et al. (2018) FGF21 underlies a hormetic response to metabolic stress in methylmalonic acidemia. JCI Insight. 3(23). pii: 124351. PMCID: PMC6328030 and Chandler et al. (2007) Metabolic phenotype of methylmalonic acidemia in mice and humans: the role of skeletal muscle. BMC Med Genet. 8:64. PMCID: PMC2140053.

As part of an ongoing effort to develop effective and safe AAV gene therapy for MMA, the present disclosure provides a new series of gene therapy vectors that are novel, and highly effective in vivo, in mouse models of MMA. Some have been engineered to be devoid of remnant AAV sequences that theoretically could increase genotoxic risk to humans. Utilized in some embodiments of the new series of gene therapy vectors is a synthetic codon-optimized human MMUT (a.k.a MUT) gene synMUT₁ to improve the expression of methylmalonyl-CoA mutase to create vectors that express the human MMUT protein in a more efficient fashion. This synthetic gene synMUT₁ (SEQ ID NO: 2) is translated as or more efficiently than the natural counterpart MMUT.

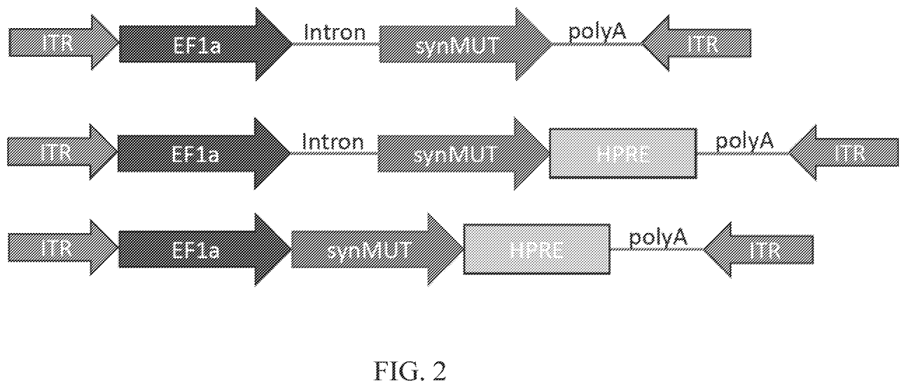

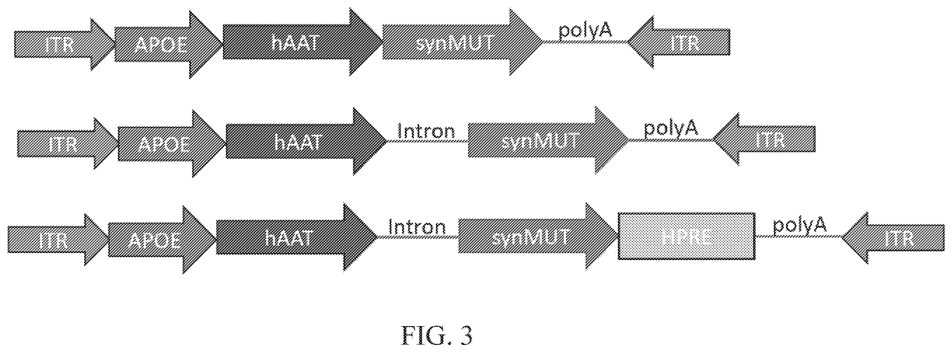

The present disclosure provides a series of new AAV vectors. In one embodiment, a synMMUT1 allele (SEQ ID NO: 2) is combined with the long human elongation factor 1 α (EF1L) promoter (SEQ ID NO: 6) (FIG. 2) or short EF1 human elongation factor 1 α (EF1S) promoter (FIG. 2) to produce vectors that express the MMUT protein at moderate levels in a global fashion (FIG. 2, SEQ ID NOs: 18, 20-21). In another embodiment, a synMMUT1 allele (SEQ ID NO: 2) is combined with the liver specific promoter from the human alpha-1 antitrypsin (hAAT) locus to produce vectors that direct MMUT protein expression in a liver specific fashion (FIG. 3, SEQ ID NO: 19). The human elongation factor 1 α cassettes variably include 130, 141 or 145 base pair AAV2 ITRs, long or shortened versions of the human elongation factor 1α (EF1α) promoter, a chimeric intron, one derived from the beta-globin locus, or a synthetic intron, the hepatitis B post-transcriptional regulatory element, and a polyadenylation signal from the bovine growth hormone or rabbit beta globin gene (FIG. 2). The liver-specific cassettes variably include short and long versions of the human apolipoprotein E gene enhancer, the human alpha 1-antitrypsin promoter, a chimeric intron or one derived from the beta-globin locus, the hepatitis B post-transcriptional regulatory element, and a polyadenylation signal from the bovine growth hormone or rabbit beta globin gene (FIG. 3). Cassettes of each construct class were cloned into plasmids that carry the ampicillin or kanamycin resistance genes, and used to prepare AAV serotype 8 and 9 vectors for testing in MMA mice. The sequences of the various elements and cassettes are listed in Table 2.

In some aspects, provided herein are host cells comprising a recombinant nucleic acid molecule, viral vector, e.g., an AAV vector, or an rAAV disclosed herein. In specific embodiments, the host cells may be suitable for the propagation of AAV.

A vast range of host cells can be used, such as bacteria, yeast, insect, mammalian cells, etc. In some embodiments, the host cell can be a cell (or a cell line) appropriate for production of recombinant AAV (rAAV), for example, a HeLa, Cos-7, HEK293, A549, BHK, Vero, RD, HT-1080, ARPE-19, or MRC-5 cell.

The recombinant nucleic acid molecules or vectors can be delivered into the host cell culture using any suitable method known in the art. In some embodiments, a stable host cell line that has the recombinant nucleic acid molecule or vector inserted into its genome is generated. In some embodiments, a stable host cell line is generated, which contains an rAAV vector described herein. After transfection of the rAAV vector to the host culture, integration of the rAAV into the host genome can be assayed by various methods, such as antibiotic selection, fluorescence-activated cell sorting, southern blot, PCR based detection, fluorescence in situ hybridization as described by Nakai et al, Nature Genetics (2003) 34, 297-302; Philpott et al, Journal of Virology (2002) 76(11): 5411-5421, and Howden et al, J Gene Med 2008; 10:42-50. Furthermore, a stable cell line can be established according to protocols well known in the art, such as those described in Clark, Kidney International Vol 61 (2002):S9-S15, and Yuan et al, Human Gene Therapy 2011 May; 22(5):613-24.

AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency.

The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Days and Bems, Clin Microbiol Rev 21(4):583-593, 2008).

The left ORF of AAV contains the Rep gene, which encodes four proteins-Rep78, Rep68, Rep52 and Rep40. The right ORF contains the Cap gene, which produces three viral capsid proteins (VP1, VP2 and VP3), and variably the AAP (accessory adapter protein). The AAV capsid contains 60 viral capsid proteins arranged into an icosahedral symmetry. VP1, VP2 and VP3 are present in a 1:1:10 molar ratio (Daya and Bems, Clin Microbiol Rev 21(4):583-593, 2008).

AAV is currently one of the most frequently used viral vectors for gene therapy. Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. Because of the advantageous features of AAV, the present disclosure contemplates the use of AAV for the recombinant nucleic acid molecules and methods disclosed herein.

AAV possesses several desirable features for a gene therapy vector, including the ability to bind and enter target cells, enter the nucleus, the ability to be expressed in the nucleus for a prolonged period of time, and low toxicity. However, the small size of the AAV genome limits the size of heterologous DNA that can be incorporated. To minimize this problem, AAV vectors have been constructed that do not encode Rep and the integration efficiency element (IEE). The ITRs are retained as they are cis signals required for packaging (Daya and Bems, Clin Microbiol Rev, 21(4):583-593, 2008).

Methods for producing rAAV suitable for gene therapy are well known in the art (see, for example, U.S. Patent Application Nos. 2012/0100606; 2012/0135515; 2011/0229971; and 2013/0072548; and Ghosh et ah, Gene Ther 13(4):321-329, 2006), and can be utilized with the recombinant nucleic acid molecules and methods disclosed herein.

In some aspects, the present disclosure provides the use of an rAAV disclosed herein for the treatment of methylmalonic acidemia (MMA), wherein the rAAV includes an AAV capsid and a vector genome packaged therein. In some embodiments, the rAAV contains a packaged genome comprising as operably linked components in 5′ to 3′ order: a 5′-ITR; a promoter sequence; optionally, an intron sequence; a partial or complete coding sequence for MMUT which may be codon optimized; optionally, a HPRE; a polyadenylation signal which can be bovine growth hormone polyA or rabbit beta-globin polyA; and a 3′-ITR. In some embodiments, the rAAV contains a packaged genome comprising as operably linked components in 5′ to 3′ order: a 5′-ITR; an enhancer sequence; a promoter sequence; optionally, an intron sequence; a partial or complete coding sequence for MMUT which may be codon optimized; optionally, a HPRE; a polyadenylation signal which can be bovine growth hormone polyA or rabbit beta-globin polyA; and a 3′-ITR. In an exemplary embodiment, the packaged genome comprises an enhancer sequence upstream of the promoter sequence, an intron downstream of the promoter, and a polyadenylation sequence upstream of the 3′-ITR. In a further exemplary embodiment, the rAAV contains a packaged genome comprising as operably linked components in 5′ to 3′ order: an AAV2 5′-ITR sequence, an EF1α promoter, a chimeric or hemoglobin subunit beta intron, a coding sequence for MMUT, optionally, a HPRE; a polyadenylation signal which can be bovine growth hormone polyA or rabbit beta-globin polyA; and a 3′-ITR. In a further exemplary embodiment, the rAAV contains a packaged genome comprising as operably linked components in 5′ to 3′ order: an AAV2 5′-ITR sequence, a APOE enhancer, a hAAT promoter, a chimeric or hemoglobin subunit beta intron, a coding sequence for MMUT, a polyadenylation signal which can be bovine growth hormone polyA or rabbit beta-globin polyA; and a 3′-ITR. In some embodiments, the coding sequence for MMUT is SEQ ID NO: 2. In some embodiments, the capsid is an AAV9 capsid. In some embodiments, the capsid is an AAV8 capsid.

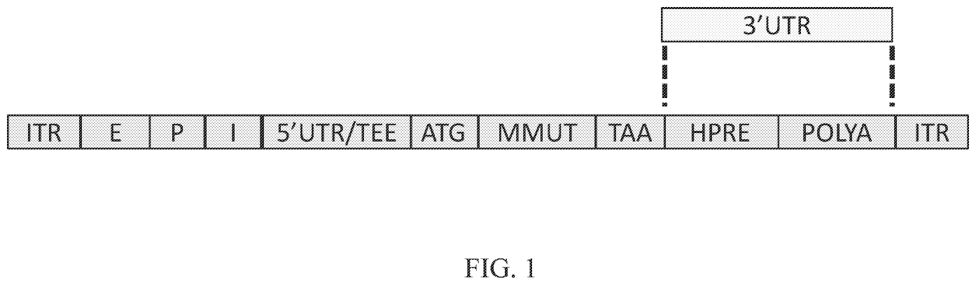

An illustrative diagram showing an exemplary packaged vector genome construct for the expression of MMUT is provided in FIGS. 1 and 2, which shows in 5′ to 3′ order: a) a 5′-inverted terminal repeat sequence (5′-ITR) sequence; (b) a promoter sequence; (c) optionally, an intron sequence; (d) optionally, a translational enhancer element (TEE); (e) partial or complete coding sequence for MMUT which may be codon optimized; (f) optionally, a Hepatitis B virus posttranscriptional regulatory element; (g) a polyadenylation sequence which can be bovine growth hormone polyA or rabbit beta-globin polyA; and (h) a 3′-inverted terminal repeat sequence (3′-ITR) sequence.

A further illustrative diagram showing an exemplary packaged vector genome construct for the expression of MMUT is provided in FIG. 3, which shows in 5′ to 3′ order: a 5′-ITR; an enhancer sequence; a promoter sequence; optionally, an intron sequence; a partial or complete coding sequence for MMUT which may be codon optimized; optionally, a HPRE; a polyadenylation signal which can be bovine growth hormone polyA or rabbit beta-globin polyA; and a 3′-ITR.

In certain embodiments, the present disclosure provides two broad classes of AAV8 and 9 vectors to treat hereditary methylmalonic acidemia caused by MMUT deficiency. In some embodiments, these new gene therapy vectors incorporate a constitutive (SEQ ID NO: 18) or liver-specific promoter (SEQ ID NO: 19) and are designed to express a codon-optimized MMUT transgene in the liver, and throughout the body. One class of vector containing the full-length human elongation factor 1 a (EF1L) promoter (SEQ ID NO: 18), serotyped as AAV8 or AAV9, was further studied in 4 distinct mouse models that recapitulate the neonatal, juvenile and adult spectrum of MMA, including one that replicates the detrimental effects of environmental (dietary) stress commonly experienced by human patients. Delivery routes and dose ranges for therapeutic effects were tested and are provided in the present disclosure.

Compositions comprising the rAAV disclosed herein and a pharmaceutically acceptable carrier are provided by the present disclosure. Suitable pharmaceutical formulations for administration of rAAV can be found, for example, in U.S. Patent Application Publication No. 2012/0219528. The pharmaceutically acceptable carriers (vehicles) useful in this disclosure are conventional. Remington's Pharmaceutical Sciences, by E. W. Martin, Mack Publishing Co., Easton, Pa., 22nd Edition (2012), describes compositions and formulations suitable for pharmaceutical delivery of one or more therapeutic compounds, molecules or agents.

As highlighted in the preceding paragraph, the present disclosure relates in some aspects to pharmaceutical compositions comprising an rAAV of the invention. In some embodiments, the pharmaceutical composition comprises a pharmaceutically acceptable carrier or excipient. In some embodiments, the pharmaceutical composition is formulated for subcutaneous, intramuscular, intradermal, intraperitoneal, or intravenous administration. In an exemplary embodiment, the pharmaceutical composition is formulated for intravenous administration.

In some embodiments, the rAAV is formulated in a buffer/carrier suitable for infusion in human subjects. The buffer/carrier should include a component that prevents the rAAV from sticking to the infusion tubing but does not interfere with the rAAV binding activity in vivo. Various suitable solutions may include one or more of a buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration. The pH may be in the range of 6.5 to 8.5, or 7 to 8.5, or 7.5 to 8. A suitable surfactant, or combination of surfactants, may be selected from among Poloxamers, i.e., nonionic triblock copolymers composed of a central hydrophobic chain of polyoxypropylene 10 (poly (propylene oxide)) flanked by two hydrophilic chains of polyoxyethylene (polyethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol.

In yet another aspect, the present disclosure provides methods of treating MMA in a human subject comprising administering to the human subject a therapeutically effective amount of at least one rAAV disclosed herein.

In one embodiment, the present disclosure provides a method of treating MMA comprising administering an rAAV that includes an AAV capsid and a vector genome packaged therein, wherein the vector genome comprises a partial or complete coding sequence for MMUT or an isoform thereof, or a functional fragment or functional variant thereof.

In certain embodiments, the present disclosure provides methods of treating MMA in a human subject comprising administering to a human subject diagnosed with at least one mutation in MMUT a therapeutically effective amount of at least one rAAV disclosed herein. In one embodiment, the present disclosure provides a method of treating MMA in a human subject diagnosed with at least one mutation in MMUT comprising administering an rAAV that includes an AAV capsid and a vector genome packaged therein, wherein the vector genome comprises a partial or complete coding sequence for MMUT or an isoform thereof, or a functional fragment or functional variant thereof. In some embodiments, the coding sequence for MMUT is SEQ ID NO: 2. In some embodiments, the capsid is an AAV9 capsid. In other embodiments, the capsid is an AAV8 capsid.

Any suitable method or route can be used to administer an rAAV or an rAAV-containing composition described herein. Routes of administration include, for example, systemic, oral, inhalation, intranasal, intratracheal, intraarterial, intraocular, intravenous, intramuscular, subcutaneous, intradermal, and other parental routes of administration. In some embodiments, the rAAV, a composition comprising an rAAV, or a composition comprising multiple rAAVs are administered intravenously.

Formulations suitable for oral administration can comprise or consist of (a) liquid solutions, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard or softshelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can include a flavor, usually sucrose and acacia or tragacanth, as well as pastilles comprising the material in an inert base, such as gelatin and glycerin, or sucrose and acacia, emulsions, gels, and the like containing, in addition to, such excipients as are known in the art.

Formulations suitable for parenteral administration include aqueous and nonaqueous isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. Administration can be in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-p-aminopropionates, and 2-alkyl-imidazoline quaternary ammonium salts, and (e) mixtures thereof.

In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Injectable formulations are contemplated. The requirements for effective pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Toissel, 4th ed., pages 622-630 (1986)).

The specific dose administered can be a uniform dose for each patient, for example, 1.0e11-1.0e14 genome copies (GC) of virus per patient. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can also be adjusted as the progress of the disease is monitored.

In some embodiments, the rAAV is administered at a dose of, e.g., about 1.0×10¹¹ genome copies per kilogram of patient body weight (GC/kg) to about 1×10¹⁴ GC/kg, about 5×10¹¹ genome copies per kilogram of patient body weight (GC/kg) to about 5×10¹³ GC/kg, or about 1×10¹² to about 1×10¹³ GC/kg, as measured by qPCR or digital droplet PCR (ddPCR).

In some embodiments, the rAAV is administered at a dose of about 1×10¹² to about 1×10¹³ genome copies (GC)/kg. In some embodiments, the rAAV is administered at a dose of about 1.1×10¹¹, about 1.3×10¹¹, about 1.6×10¹¹, about 1.9×10¹¹, about 2×10¹¹, about 2.5×10¹¹, about 3.0×10¹¹, about 3.5×10¹¹, about 4.0×10¹¹, about 4.5×10¹¹, about 5.0×10¹¹, about 5.5×10¹¹, about 6.0×10¹¹, about 6.5×10¹¹, about 7.0×10¹¹, about 7.5×10¹¹, about 8.0×10¹¹, about 8.5×10¹¹, about 9.0×10¹¹, about 9.5×10¹¹, about 1.0×10¹², about 1.5×10¹², about 2.0×10¹², about 2.5×10¹², about 3.0×10¹², about 3.5×10¹², about 4.0×10¹², about 4.5×10¹², about 5.0×10¹², about 5.5×10¹², about 6.0×10¹², about 6.5×10¹², about 7.0×10¹², about 7.5×10¹², about 8.0×10¹², about 8.5×10¹², about 9.0×10¹², about 9.5×10¹², about 1.0×10¹³, about 1.5×10¹³, about 2.0×10¹³, about 2.5×10¹³, about 3.0×10¹³, about 3.5×10¹³, about 4.0×10¹³, about 4.5×10¹³, about 5.0×10¹³, about 5.5×10¹³, about 6.0×10¹³, about 6.5×10¹³, about 7.0×10¹³, about 7.5×10¹³, about 8.0×10¹³, about 8.5×10¹³, about 9.0×10¹³, about 9.5×10¹³ genome copies (GC)/kg. The rAAV can be administered in a single dose, or in multiple doses (such as 2, 3, 4, 5, 6, 7, 8, 9, 10 or more doses) as needed for the desired therapeutic results.

Doses may be given once or more times weekly, monthly or yearly, or even once every 2 to 20 years. For example, each dose may be given at minimum of 1 week apart, 2 weeks apart, 3 weeks apart, a month apart, 3 months apart, 6 months apart, or 1 year apart. Persons of ordinary skill in the art can easily estimate repetition rates for dosing based on measured residence times and concentrations of the targetable construct or complex in bodily fluids or tissues.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the present disclosure, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within the present disclosure, embodiments have been described and depicted in a way that enables a clear and concise disclosure to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including” is intended merely to illustrate better the present invention and does not pose a limitation on the scope of the invention unless claimed. No language in the specification should be construed as indicating any non-claimed element as essential to the practice of the present invention.

### EXAMPLES

It shall be noted that the preceding are merely examples of embodiments. Other exemplary embodiments are apparent from the entirety of the description herein. It will also be understood by one of ordinary skill in the art that each of these embodiments may be used in various combinations with the other embodiments provided herein.

The following examples further illustrate the invention but, of course, should not be construed as in any way limiting its scope.

### Example 1

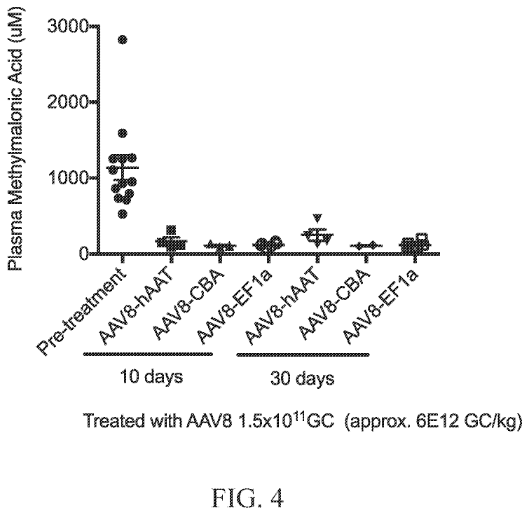

This example demonstrates the results of two classes of AAV vectors encoding the codon-optimized synMMUT1 gene (SEQ ID NO: 2). It has been recognized, via gene therapy studies and transgenic animal experiments, that the restoration of gene expression in the liver is of utmost importance to correct the phenotype of MMA. In this example, two new classes of AAV vectors encoding synMUT1 (SEQ ID NO: 2) under the control of the EF1 (constitutive, low/moderate expression) (SEQ ID NO: 18) or hAAT (liver) (SEQ ID NO: 19) were compared to a CBA (constitutive, high expression) promoter vector. In one study, the cassettes were packaged using an AAV8 or AAV9 capsid to create AAV8-CBA-synMMUT1, AAV8-EF1L-synMMUT1, and AAV8-hAAT-synMMUT1 and delivered to MMA mice by retroorbital injection. AAV8-CBA-synMMUT1 was prepared as described in U.S. Pat. No. 9,944,918. The mice used in this study, Mmut^{-/-}; Tg^{MckMut}, were chosen to allow the comparison of relative hepatic correction. These mice express the mouse Mmut enzyme in the skeletal muscle and the recapitulate hepato-renal deficiency of human MMA, clinically with growth retardation and fragility, and biochemically through massive elevations of methylmalonic acid as well as impaired whole-body capacity to oxidize 1-C-13 propionic acid. These mice were disclosed in Manoli et al., (2018) FGF21 underlies a hormetic response to metabolic stress in methylmalonic acidemia. JCI Insight. 3(23):e124351. Published 2018 Dec. 6. doi:10.1172/jci.insight.124351 In prior work, it was demonstrated that the hepatic correction of Mmut deficiency, by transgenesis (Manoli et al. (2013) Targeting proximal tubule mitochondrial dysfunction attenuates the renal disease of methylmalonic acidemia. Proc Natl Acad Sci USA. 110(33):13552-13557. doi:10.1073/pnas.1302764110) or liver directed AAV gene therapy (Carrillo Carrasco et al. (2012) Liver-directed recombinant adeno-associated viral gene delivery rescues a lethal mouse model of methylmalonic acidemia and provides long-term phenotypic correction. Hum Gene Ther. 2010; 21(9):1147-1154. doi:10.1089/hum.2010.008), can correct the lethal phenotype seen in full Mmut knock-out mice (Mut^{-/-}). Therefore, the use of systemic gene therapy and the vectors described herein was used to assay the effects of hepatic correction on the phenotype of the Mmut^{-/-}; Tg^{MckMut} mice. In brief, 3-4 adult Mmut^{-/-}; Tg^{MckMut} mice were injected with either AAV8-HAAT-synMUT1, AAV8-CBA-synMUT1, or AAV8-EF1L-synMUT1. 1.5e11 genome copies (GC) of each vector, roughly equating to a dose of 6e12 GC/kg, was delivered by retro-orbital injection to individual Mmut^{-/-}; Tg^{MckMut} mice. Vectors were prepared as previously described in Chandler R J, Venditti C P. (2010) Long-term rescue of a lethal murine model of methylmalonic acidemia using adeno-associated viral gene therapy. Mol Ther. 18(1):11-6 PMCID: PMC2839224 and in Chandler R J, Venditti C P. (2012) Pre-clinical efficacy and dosing of an AAV8 vector expressing human methylmalonyl-CoA mutase in a murine model of methylmalonic acidemia (MMA). Mol Genet Metab. 107(3):617-9. PMCID: PMC3522145 using techniques that are well know to practioners of the art. The plasma methylmalonic acid concentration after 10 and 30 days was measured, and compared to historical controls. As can be seen in FIG. 4, each construct induced an equivalent biochemical response in the Mmut^{-/-}; Tg^{MckMut} mice, suggesting that the efficacy of hepatic gene expression provided by each construct was therapeutic, and furthermore, that the relatively weaker EF1α promoter was highly effective in liver-directed gene therapy for the correction of hepatic Mmut deficiency.

### Example 2

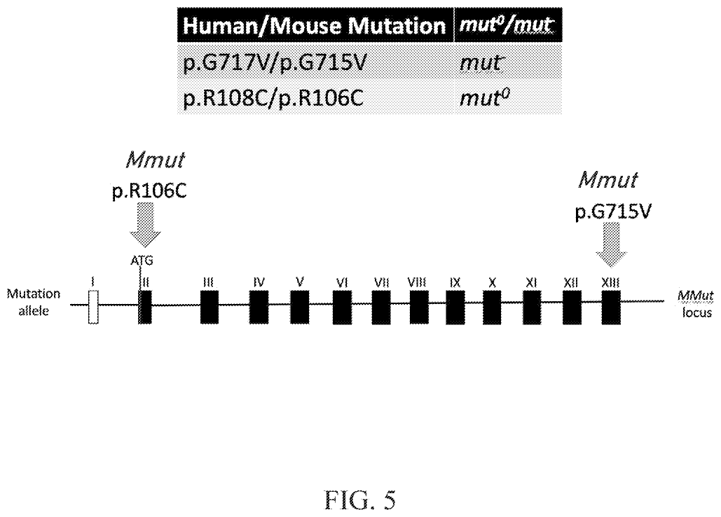

This example demonstrates the utility of AAV8-EF1L-synMUT1 as an effective gene therapy vector for systemic treatment of knock-in mouse models of MMA. In order to create new mouse models of MMA to resemble the pathogenic mutations seen in patients, genome editing was used to generate homologous mutant Mmut alleles (FIG. 5). These alleles recapitulate missense mutations seen in multiple patients with the disorder. MMUTp.R108C and MMUTp.G717V MMA patients display variability in age at disease onset, suggesting that mouse models with these mutations might express Mmut with sufficient activity to rescue from neonatal lethality or produce an intermediate phenotype of decreased survival without immediate lethality. Of note and emphasis is the fact that there are no transgene cassettes or other alternations to the Mmut locus in these new mouse models.

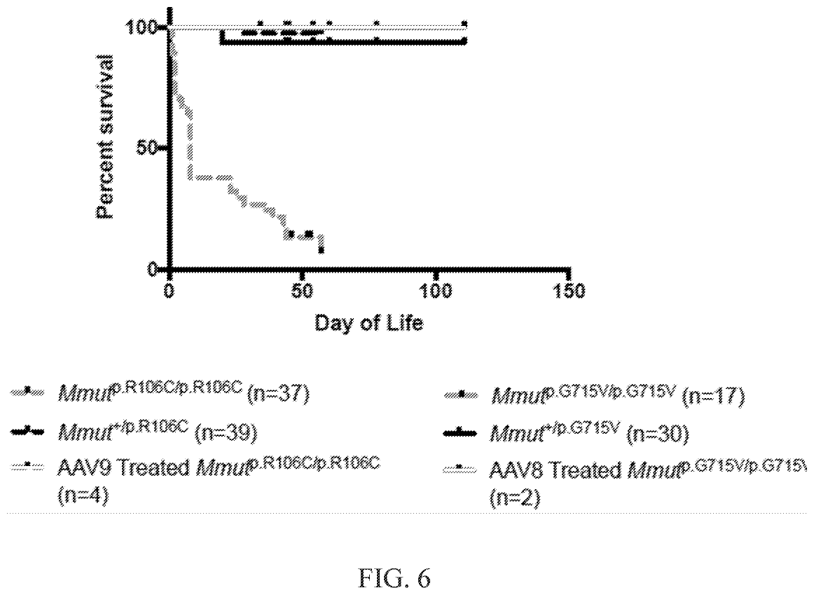

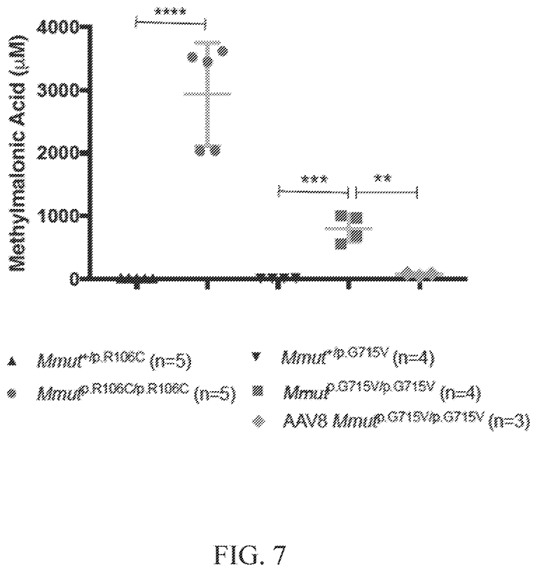

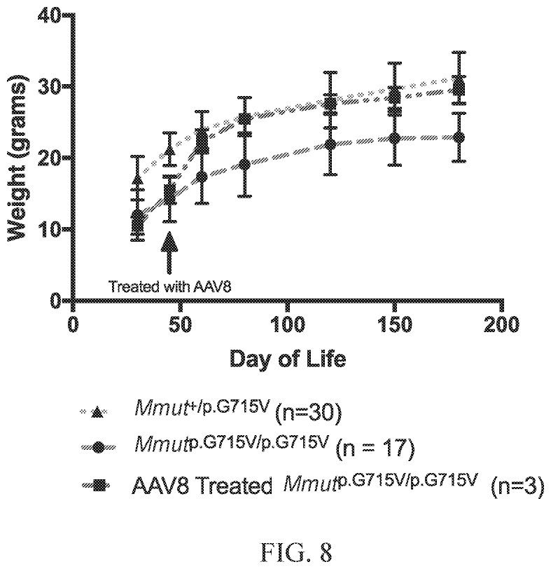

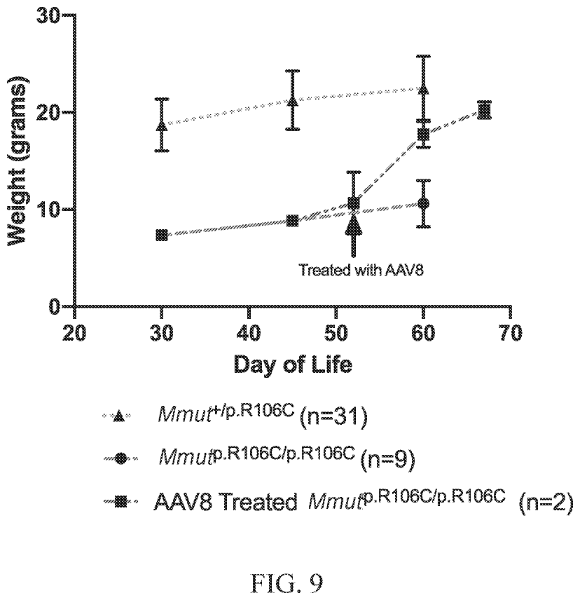

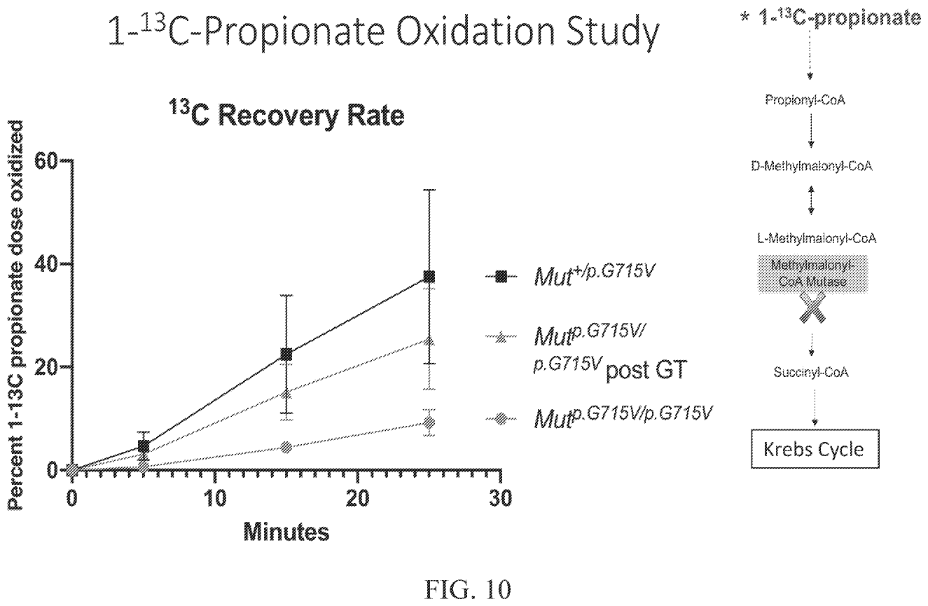

Mmutp.R106C/p.R106C exhibit severe lethality compared to Mmutp.G715V/p.G715V mice (FIG. 6). Both models respond to AAV gene therapy. When the EF1L synMMUT1 cassette was produced as an AAV9 vector (EF1L AAV9), prepared as described in Senac J S, Chandler R J, Sysol J R, Li L, Venditti C P. (2012) Gene Therapy in a murine model of Methylmalonic Acidemia (MMA) using rAAV9 mediated gene delivery. Gene Ther. 19(4):385-91. PMCID: PMC338206 PMC3522145 using techniques that are well know to practioners of the art, and delivered to neonatal Mmutp.R106C/p.R106C mutant mice at a dose of 1e11 GC/pup via retroorbital injection, the treatment afforded complete rescue from lethality and increased survival past weaning in the Mmutp.R106C/p.R106C mutant mice (FIG. 6). In addition, 1e13 GC/kg of AAV8 EF1L was delivered by the retro-orbital route to adult Mmutp.G715V/p.G715V or Mmutp.R106C/p.R106C mutant mice. Improved metabolites and growth (FIGS. 7, 8 and 9), reduced levels of plasma methymalonic acid (FIG. 7, Mmutp.G715V/p.G715V mice), and increased 1 C-13 propionate oxidative capacity (FIG. 10, Mmutp.G715V/p.G715V mice) were noted. This example shows that one lead candidate AAV vector, EF1L synMMUT1 (SEQ ID NO: 18), has broad efficacy in mouse models of MMA and can rapidly induce phenotypic and biochemical responses by transducing the liver, as well as other tissues, when serotyped as either AAV8 or AAV9 capsids.

### Example 3

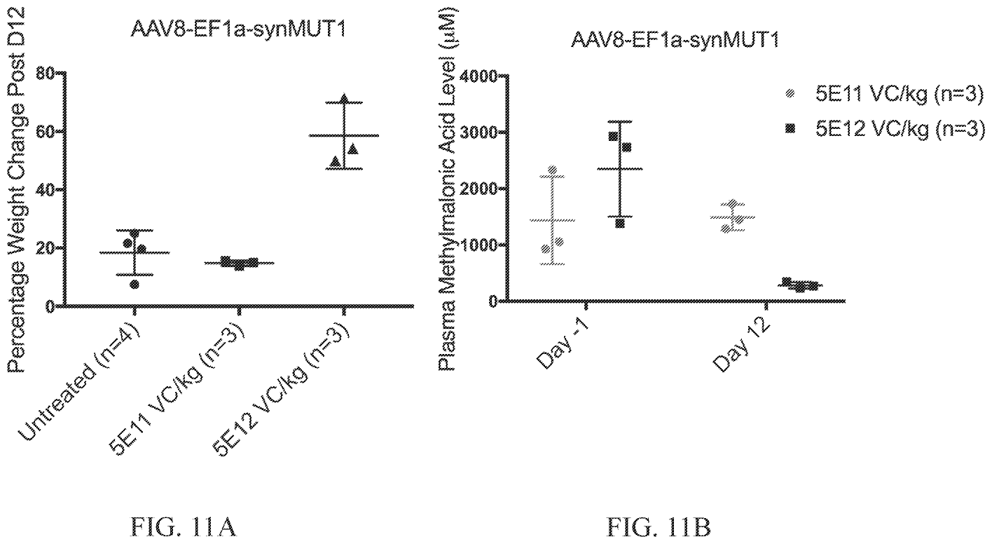

FIGS. 11A and 11B detail studies in Mmut^{p.G715V/p.G715V} mice aimed at predicting a therapeutic range for dosing patients with MMA. In brief, adult Mmut^{p.G715V/p.G715V} mice (n=3) were injected with AAV8 EF1L synMMUT1 gene therapy at a dose of either 5e12 GC/kg or 5e11 GC/kg and compared to untreated Mmut^{p.G715V/p.G715V} mice. The mice treated with 5e12 GC/kg gained more weight and had much lower levels of methylmalonic acid in the blood after gene therapy, suggesting that the lower range of the dose for a patient should approximate 5e12 GC/kg or more for a robust clinical and biochemical response after AAV8 EF1L synMMUT1 gene therapy.

### Example 4

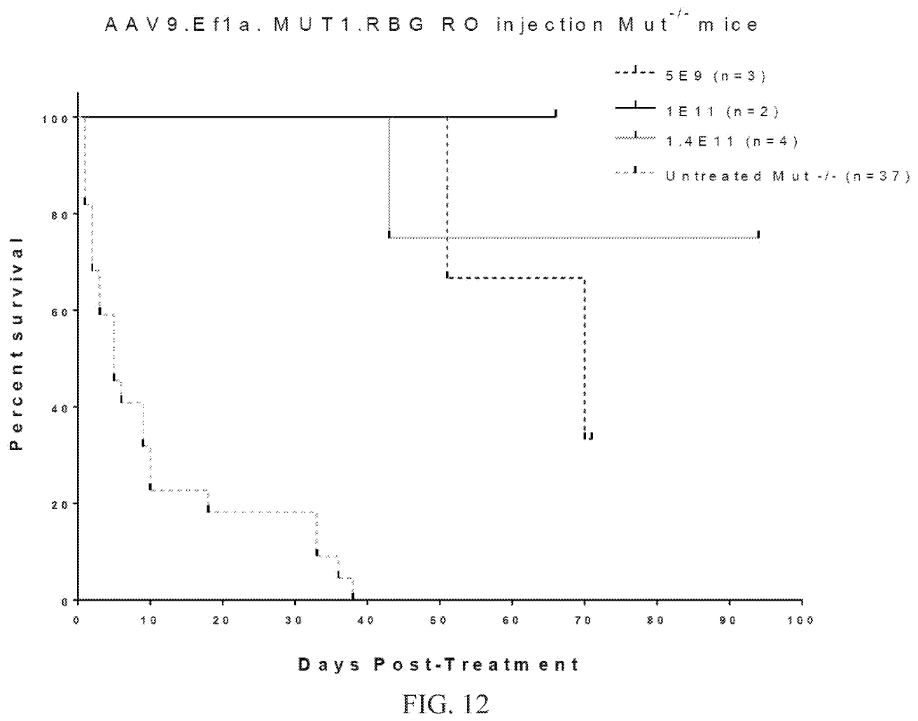

This example describes the use of AAV9 EF1L synMMUT1 gene therapy to rescue the most severe MMA mouse model described to date, the full Mmut knock-out or Mmut^{-/-} mice. These animals display near immediate neonatal lethality but can be rescued by AAV vectors that contain potent enhancer and promoters, such as the CMV enhanced, chicken beta actin promoter (Chandler and Venditti, Mol Ther 2009). As can be seen in FIG. 12, systemic delivery of AAV9 EF1L synMMUT1 at doses ranging from 5e9 GC/pup to 1.4e11 GC/pup were effective in rescuing the Mmut^{-/-} mice from death, and provided long term growth and stability. The comparison was to historical controls. Thus, this vector and serotype, in ranges noted with other enhanced promoters (Chandler R J, Venditti C P. Pre-clinical efficacy and dosing of an AAV8 vector expressing human methylmalonyl-CoA mutase in a murine model of methylmalonic acidemia (MMA). Mol Genet Metab. 2012; 107(3):617-619), appeared equally effective for neonatal rescue.

### Example 5

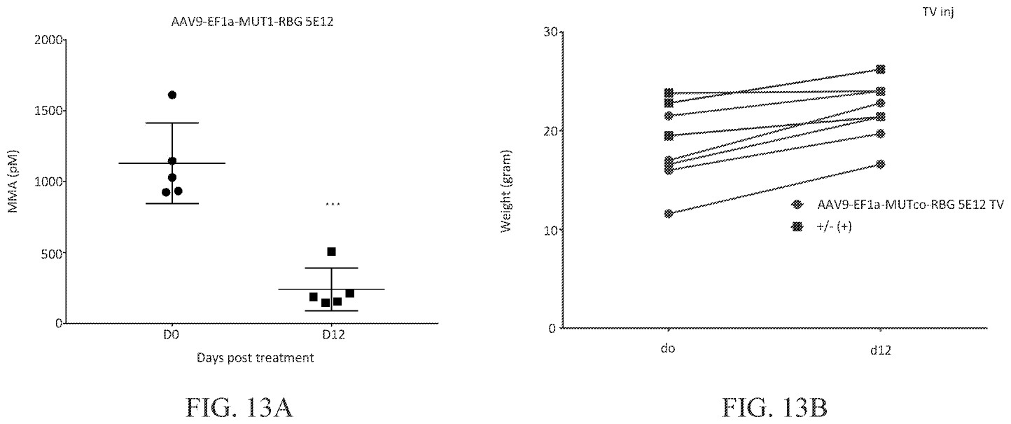

This example (FIGS. 13A-13B) describes the use of AAV9 EF1L synMMUT1 gene therapy to generate a rapid biochemical and clinical response in Mmut^{-/-}; Tg^{MckMut} mice (n=5) to a 5e12 GC/kg dose of AAV9 EF1L synMMUT1 delivered by tail vein injection. Metabolites (methymalonic acid levels in the plasma) and clinical response (weight gain) were monitored before the injection and 12 days after treatment. A robust response was noted, and was comparable to the response seen after treatment of Mmut^{-/-}; Tg^{MckMut} mice with AAV8 hAAT synMMUT1, AAV8 EF1L synMMUT1, and AAV8 CBA synMMUT1 (FIG. 3). Thus, this vector and serotype, in ranges noted with other enhanced promoters, appeared equally effective for liver directed gene therapy for MMA, using the tail vein delivery route.

### Example 6

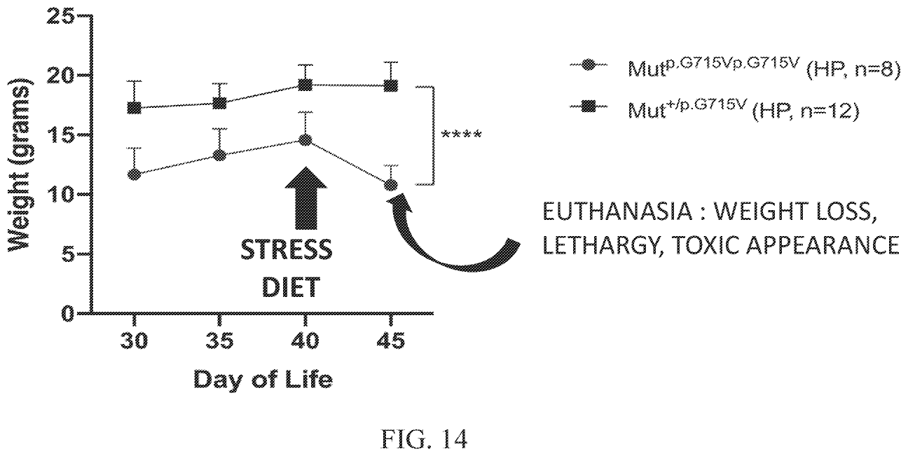

This example shows how AAV9 EF1L synMMUT1 gene therapy can protect Mmut^{p.G715V/p.G715V} mice from a challenge to the propionyl-CoA oxidation pathway through metabolic stress, via increased branched chain amino acid intake in the diet. The production of toxic disease related metabolites in the patients is well recognized to derive from the dietary intake of branched chain amino acids, such as valine and isoleucine. Therefore, we developed a challenge regimen to stress adult Mmut^{p.G715V/p.G715V} mice (n=8) with a high protein challenge. The mutant mice and control littermates were maintained on a regular chow diet and switched to 70% by weight casein chow. This diet massively increases the intake of valine and isoleucine, the main precursors of methylmalonic acid. Within 5 days of dietary challenge, only the Mmut^{p.G715V/p.G715V} animals lost >20% of their body weight, became obtunded, had increased levels of methylmalonic acid in the blood, and needed to be euthanized (FIG. 14). The heterozygous littermates thrived and gained weight on the same diet.

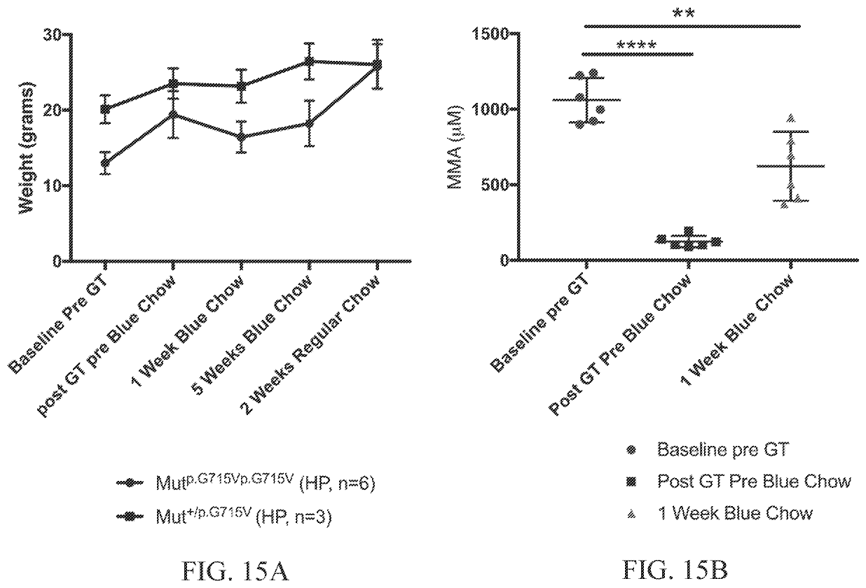

Next, adult Mmut^{p.G715V/p.G715V} mice (n=6) were administered AAV9 EF1L synMMUT1 at a dose of 5e12 GC/kg and after two weeks, were exposed to the same high protein diet (70% by weight casein chow-labeled blue chow). While similarly stressed untreated Mmut^{p.G715V/p.G715V} mice perish within a week of exposure to a high protein challenge, Mmut^{p.G715V/p.G715V} mice treated with AAV9 EF1L synMMUT1 tolerated a high protein diet/metabolic challenge as proven by extended survival, but did manifest slowed growth (FIG. 15A) and increased metabolites (FIG. 15B). After resumption of a regular diet, the AAV9 EF1L synMMUT1 treated Mmut^{p.G715V/p.G715V} mice rapidly recovered growth to the level of littermate controls. Hence, AAV9 EF1L synMMUT1 gene therapy, administered at a modest dose of 5e12 GC/kg was fully protective against diet induced fatality, and durable.

### Example 7

This example shows how transgene expression after AAV9 EF1L synMMUT1 gene therapy can be detected using the unique sequence of synMMUT1 in an RNA in situ hybridization assay.

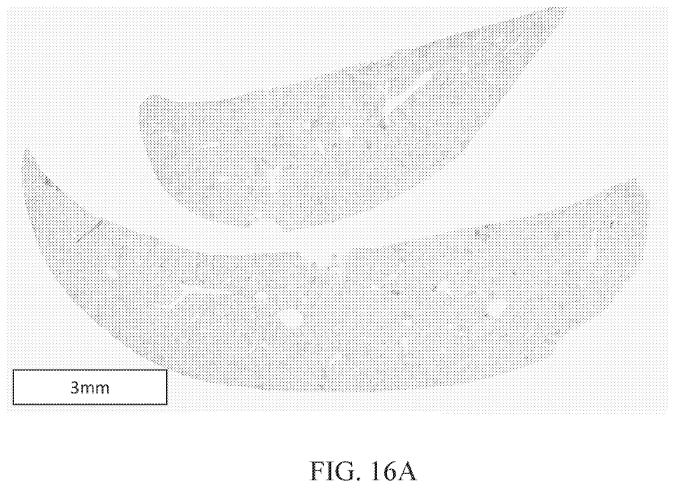

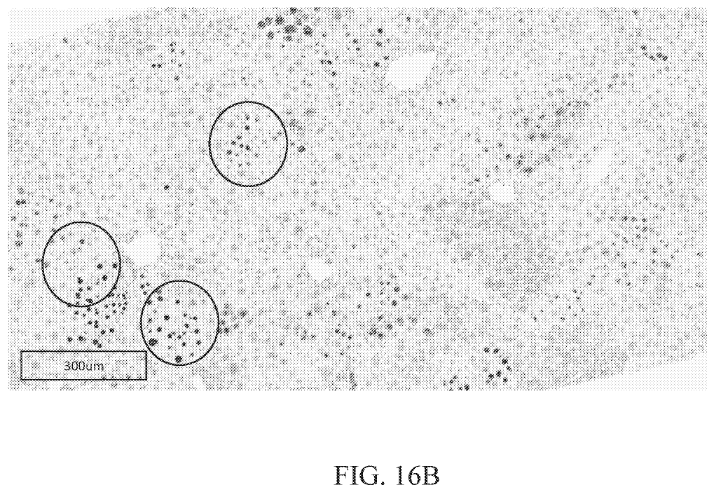

In this study, a representative Mmut^{p.R106C/p.R106C} mouse was treated at birth with 1e11GC AAV9 EF1L synMMUT1 and then sacrificed at 6 months of age. The liver was removed and fixed in 4% formalin and processed into paraffin blocks. Five-micron sections were cut and stained with a probe designed to detect synMMUT1 using the RNAScope 2.5 HD Assay-Brown (ACDBio 322300) following the manufacturer's instructions. Slide images were captured using the Zeiss AxioScan Z1 slide scanner and analyzed using Image Pro premier 3D version 9.3 by Media Cybernetics. FIG. 16A shows a 10× magnification of a representative liver section, where dark staining cells express synMMUT1. A higher 40× magnification allows the precise detection of individual cells (circled) that express synMMUT1 over the background of endogenous Mmut, with concomitant visualization of the hepatic cytoarchitecture.

Hence, this example demonstrates the utility of a novel synthetic MMUT expressing AAV vector to enable precise nucleic acid detection, at the cellular level, as might be needed in future human studies if a liver biopsy was needed to ascertain AAV9 EF1L synMMUT1 transgene expression or for longitudinal monitoring after human gene therapy.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Also, everywhere “comprising” (or its equivalent) is recited, the “comprising” is considered to incorporate “consisting essentially of” and “consisting of” Thus, an embodiment “comprising” (an) element(s) supports embodiments “consisting essentially of” and “consisting of” the recited element(s). Everywhere “consisting essentially of” is recited is considered to incorporate “consisting of” Thus, an embodiment “consisting essentially of” (an) element(s) supports embodiments “consisting of” the recited element(s). “Consisting essentially of” limits the scope of a claim to the specified materials or steps and those that do not materially affect the basic and novel characteristic(s) of the claim.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

TABLE 2
SEQ ID NO: 1. WT MMUT
ATGTTAAGAGCTAAGAATCAGCTTTTTTTACTTTCACCTCATTACCTGAG
GCAGGTAAAAGAATCATCAGGCTCCAGGCTCATACAGCAACGACTTCTAC
ACCAGCAACAGCCCCTTCACCCAGAATGGGCTGCCCTGGCTAAAAAGCAG
CTGAAAGGCAAAAACCCAGAAGACCTAATATGGCACACCCCGGAAGGGAT
CTCTATAAAACCCTTGTATTCCAAGAGAGATACTATGGACTTACCTGAAG
AACTTCCAGGAGTGAAGCCATTCACACGTGGACCATATCCTACCATGTAT
ACCTTTAGGCCCTGGACCATCCGCCAGTATGCTGGTTTTAGTACTGTGGA
AGAAAGCAATAAGTTCTATAAGGACAACATTAAGGCTGGTCAGCAGGGAT
TATCAGTTGCCTTTGATCTGGCGACACATCGTGGCTATGATTCAGACAAC
CCTCGAGTTCGTGGTGATGTTGGAATGGCTGGAGTTGCTATTGACACTGT
GGAAGATACCAAAATTCTTTTTGATGGAATTCCTTTAGAAAAAATGTCAG
TTTCCATGACTATGAATGGAGCAGTTATTCCAGTTCTTGCAAATTTTATA
GTAACTGGAGAAGAACAAGGTGTACCTAAAGAGAAGCTTACTGGTACCAT
CCAAAATGATATACTAAAGGAATTTATGGTTCGAAATACATACATTTTTC
CTCCAGAACCATCCATGAAAATTATTGCTGACATATTTGAATATACAGCA
AAGCACATGCCAAAATTTAATTCAATTTCAATTAGTGGATACCATATGCA
GGAAGCAGGGGCTGATGCCATTCTGGAGCTGGCCTATACTTTAGCAGATG
GATTGGAGTACTCTAGAACTGGACTCCAGGCTGGCCTGACAATTGATGAA
TTTGCACCAAGGTTGTCTTTCTTCTGGGGAATTGGAATGAATTTCTATAT
GGAAATAGCAAAGATGAGAGCTGGTAGAAGACTCTGGGCTCACTTAATAG
AGAAAATGTTTCAGCCTAAAAACTCAAAATCTCTTCTTCTAAGAGCACAC
TGTCAGACATCTGGATGGTCACTTACTGAGCAGGATCCCTACAATAATAT
TGTCCGTACTGCAATAGAAGCAATGGCAGCAGTATTTGGAGGGACTCAGT
CTTTGCACACAAATTCTTTTGATGAAGCTTTGGGTTTGCCAACTGTGAAA
AGTGCTCGAATTGCCAGGAACACACAAATCATCATTCAAGAAGAATCTGG
GATTCCCAAAGTGGCTGATCCTTGGGGAGGTTCTTACATGATGGAATGTC
TCACAAATGATGTTTATGATGCTGCTTTAAAGCTCATTAATGAAATTGAA
GAAATGGGTGGAATGGCCAAAGCTGTAGCTGAGGGAATACCTAAACTTCG
AATTGAAGAATGTGCTGCCCGAAGACAAGCTAGAATAGATTCTGGTTCTG
AAGTAATTGTTGGAGTAAATAAGTACCAGTTGGAAAAAGAAGACGCTGTA
GAAGTTCTGGCAATTGATAATACTTCAGTGCGAAACAGGCAGATTGAAAA
ACTTAAGAAGATCAAATCCAGCAGGGATCAAGCTTTGGCTGAACGTTGTC
TTGCTGCACTAACCGAATGTGCTGCTAGCGGAGATGGAAATATCCTGGCT
CTTGCAGTGGATGCATCTCGGGCAAGATGTACAGTGGGAGAAATCACAGA
TGCCCTGAAAAAGGTATTTGGTGAACATAAAGCGAATGATCGAATGGTGA
GTGGAGCATATCGCCAGGAATTTGGAGAAAGTAAAGAGATAACATCTGCT
ATCAAGAGGGTTCATAAATTCATGGAACGTGAAGGTCGCAGACCTCGTCT
TCTTGTAGCAAAAATGGGACAAGATGGCCATGACAGAGGAGCAAAAGTTA
TTGCTACAGGATTTGCTGATCTTGGTTTTGATGTGGACATAGGCCCTCTT
TTCCAGACTCCTCGTGAAGTGGCCCAGCAGGCTGTGGATGCGGATGTGCA
TGCTGTGGGCATAAGCACCCTCGCTGCTGGTCATAAAACCCTAGTTCCTG
AACTCATCAAAGAACTTAACTCCCTTGGACGGCCAGATATTCTTGTCATG
TGTGGAGGGGTGATACCACCTCAGGATTATGAATTTCTGTTTGAAGTTGG
TGTTTCCAATGTATTTGGTCCTGGGACTCGAATTCCAAAGGCTGCCGTTC
AGGTGCTTGATGATATTGAGAAGTGTTTGGAAAAGAAGCAGCAATCTGTA
TAA
SEQ ID NO: 2 synMMUT1
ATGCTGAGAGCCAAAAACCAGCTGTTCCTGCTGAGCCCCCACTATCTGAGACAG
GTCAAAGAAAGTTCCGGGAGTAGACTGATCCAGCAGAGACTGCTGCACCAGCAG
CAGCCACTGCATCCTGAGTGGGCCGCTCTGGCCAAGAAACAGCTGAAGGGCAAA
AACCCAGAAGACCTGATCTGGCACACTCCAGAGGGGATTTCAATCAAGCCCCTG
TACAGCAAAAGGGACACTATGGATCTGCCAGAGGAACTGCCAGGAGTGAAGCCT
TTCACCCGCGGACCTTACCCAACTATGTATACCTTTCGACCCTGGACAATTCGGC
AGTACGCCGGCTTCAGTACTGTGGAGGAATCAAACAAGTTTTATAAGGACAACA
TCAAGGCTGGACAGCAGGGCCTGAGTGTGGCATTCGATCTGGCCACACATCGCG
GCTATGACTCAGATAATCCCAGAGTCAGGGGGGACGTGGGAATGGCAGGAGTCG
CTATCGACACAGTGGAAGATACTAAGATTCTGTTCGATGGAATCCCTCTGGAGAA
AATGTCTGTGAGTATGACAATGAACGGCGCTGTCATTCCCGTGCTGGCAAACTTC
ATCGTCACTGGCGAGGAACAGGGGGTGCCTAAGGAAAAACTGACCGGCACAATT
CAGAACGACATCCTGAAGGAGTTCATGGTGCGGAATACTTACATTTTTCCCCCTG
AACCATCCATGAAAATCATTGCCGATATCTTCGAGTACACCGCTAAGCACATGCC
CAAGTTCAACTCAATTAGCATCTCCGGGTATCATATGCAGGAAGCAGGAGCCGA
CGCTATTCTGGAGCTGGCTTACACCCTGGCAGATGGCCTGGAATATTCTCGAACC
GGACTGCAGGCAGGCCTGACAATCGACGAGTTCGCTCCTAGACTGAGTTTCTTTT
GGGGAATTGGCATGAACTTTTACATGGAGATCGCCAAGATGAGGGCTGGCCGGA
GACTGTGGGCACACCTGATCGAGAAGATGTTCCAGCCTAAGAACTCTAAGAGTC
TGCTGCTGCGGGCCCATTGCCAGACATCCGGCTGGTCTCTGACTGAACAGGACCC
ATATAACAATATTGTCAGAACCGCAATCGAGGCAATGGCAGCCGTGTTCGGAGG
AACCCAGAGCCTGCACACAAACTCCTTTGATGAGGCCCTGGGGCTGCCTACCGTG
AAGTCTGCTAGGATTGCACGCAATACACAGATCATTATCCAGGAGGAATCCGGA
ATCCCAAAGGTGGCCGATCCCTGGGGAGGCTCTTACATGATGGAGTGCCTGACA
AACGACGTGTATGATGCTGCACTGAAGCTGATTAATGAAATCGAGGAAATGGGG
GGAATGGCAAAGGCCGTGGCTGAGGGCATTCCAAAACTGAGGATCGAGGAATGT
GCAGCTAGGCGCCAGGCACGAATTGACTCAGGAAGCGAAGTGATCGTCGGGGTG
AATAAGTACCAGCTGGAGAAAGAAGACGCAGTCGAAGTGCTGGCCATCGATAAC
ACAAGCGTGCGCAATCGACAGATTGAGAAGCTGAAGAAAATCAAAAGCTCCCGC
GATCAGGCACTGGCCGAACGATGCCTGGCAGCCCTGACTGAGTGTGCTGCAAGC
GGGGACGGAAACATTCTGGCTCTGGCAGTCGATGCCTCCCGGGCTAGATGCACT
GTGGGGGAAATCACCGACGCCCTGAAGAAAGTCTTCGGAGAGCACAAGGCCAAT
GATCGGATGGTGAGCGGCGCTTATAGACAGGAGTTCGGGGAATCTAAAGAGATT
ACCAGTGCCATCAAGAGGGTGCACAAGTTCATGGAGAGAGAAGGGCGACGGCCC
AGGCTGCTGGTGGCAAAGATGGGACAGGACGGACATGATCGCGGAGCAAAAGT
CATTGCCACCGGGTTCGCTGACCTGGGATTTGACGTGGATATCGGCCCTCTGTTC
CAGACACCACGAGAGGTCGCACAGCAGGCAGTCGACGCTGATGTGCACGCAGTC
GGAGTGTCCACTCTGGCAGCTGGCCATAAGACCCTGGTGCCTGAACTGATCAAA
GAGCTGAACTCTCTGGGCAGACCAGACATCCTGGTCATGTGCGGCGGCGTGATCC
CACCCCAGGATTACGAATTCCTGTTTGAGGTCGGGGTGAGCAACGTGTTCGGACC
AGGAACCAGGATCCCTAAGGCCGCAGTGCAGGTCCTGGATGATATTGAAAAGTG
TCTGGAAAAGAAACAGCAGTCAGTGTAA
SEQ ID NO: 3 MMUT
MLRAKNQLFLLSPHYLRQVKESSGSRLIQQRLLHQQQPLHPEWAALAKKQLKGKNP
EDLIWHTPEGISIKPLYSKRDTMDLPEELPGVKPFTRGPYPTMYTFRPWTIRQYAGFST
VEESNKFYKDNIKAGQQGLSVAFDLATHRGYDSDNPRVRGDVGMAGVAIDTVEDT
KILFDGIPLEKMSVSMTMNGAVIPVLANFIVTGEEQGVPKEKLTGTIQNDILKEFMVR
NTYIFPPEPSMKIIADIFEYTAKHMPKFNSISISGYHMQEAGADAILELAYTLADGLEY
SRTGLQAGLTIDEFAPRLSFFWGIGMNFYMEIAKMRAGRRLWAHLIEKMFQPKNSKS
LLLRAHCQTSGWSLTEQDPYNNIVRTAIEAMAAVFGGTQSLHTNSFDEALGLPTVKS
ARIARNTQIIIQEESGIPKVADPWGGSYMMECLTNDVYDAALKLINEIEEMGGMAKA
VAEGIPKLRIEECAARRQARIDSGSEVIVGVNKYQLEKEDAVEVLAIDNTSVRNRQIE
KLKKIKSSRDQALAERCLAALTECAASGDGNILALAVDASRARCTVGEITDALKKVF
GEHKANDRMVSGAYRQEFGESKEITSAIKRVHKFMEREGRRPRLLVAKMGQDGHD
RGAKVIATGFADLGFDVDIGPLFQTPREVAQQAVDADVHAVGVSTLAAGHKTLVPE
LIKELNSLGRPDILVMCGGVIPPQDYEFLFEVGVSNVFGPGTRIPKAAVQVLDDIEKCL
EKKQQSV
SEQ ID NO: 4 ITR 130 bp
CTGCGCGCTCGCTCGCTCACTGAGGCCGCCCGGGCAAAGCCCGGGCGTCGGGCG
ACCTTTGGTCGCCCGGCCTCAGTGAGCGAGCGAGCGCGCAGAGAGGGAGTGGCC
AACTCCATCACTAGGGGTTCCT
SEQ ID NO: 5 ITR 145 bp
TTGGCCACTCCCTCTCTGCGCGCTCGCTCGCTCACTGAGGCCGCCCGGGCAAAGC
CCGGGCGTCGGGCGACCTTTGGTCGCCCGGCCTCAGTGAGCGAGCGAGCGCGCA
GAGAGGGAGTGGCCAACTCCATCACTAGGGGTTCCT
SEQ ID NO: 6 EF1L
GGCTCCGGTGCCCGTCAGTGGGCAGAGCGCACATCGCCCACAGTCCCCGAGAAG
TTGGGGGGAGGGGTCGGCAATTGAACCGGTGCCTAGAGAAGGTGGCGCGGGGTA
AACTGGGAAAGTGATGTCGTGTACTGGCTCCGCCTTTTTCCCGAGGGTGGGGGAG
AACCGTATATAAGTGCAGTAGTCGCCGTGAACGTTCTTTTTCGCAACGGGTTTGC
CGCCAGAACACAGGTAAGTGCCGTGTGTGGTTCCCGCGGGCCTGGCCTCTTTACG
GGTTATGGCCCTTGCGTGCCTTGAATTACTTCCACCTGGCTGCAGTACGTGATTCT
TGATCCCGAGCTTCGGGTTGGAAGTGGGTGGGAGAGTTCGAGGCCTTGCGCTTAA
GGAGCCCCTTCGCCTCGTGCTTGAGTTGAGGCCTGGCCTGGGCGCTGGGGCCGCC
GCGTGCGAATCTGGTGGCACCTTCGCGCCTGTCTCGCTGCTTTCGATAAGTCTCTA
GCCATTTAAAATTTTTGATGACCTGCTGCGACGCTTTTTTTCTGGCAAGATAGTCT
TGTAAATGCGGGCCAAGATCTGCACACTGGTATTTCGGTTTTTGGGGCCGCGGGC
GGCGACGGGGCCCGTGCGTCCCAGCGCACATGTTCGGCGAGGCGGGGCCTGCGA
GCGCGGCCACCGAGAATCGGACGGGGGTAGTCTCAAGCTGGCCGGCCTGCTCTG
GTGCCTGGCCTCGCGCCGCCGTGTATCGCCCCGCCCTGGGCGGCAAGGCTGGCCC
GGTCGGCACCAGTTGCGTGAGCGGAAAGATGGCCGCTTCCCGGCCCTGCTGCAG
GGAGCTCAAAATGGAGGACGCGGCGCTCGGGAGAGCGGGCGGGTGAGTCACCC
ACACAAAGGAAAAGGGCCTTTCCGTCCTCAGCCGTCGCTTCATGTGACTCCACGG
AGTACCGGGCGCCGTCCAGGCACCTCGATTAGTTCTCGAGCTTTTGGAGTACGTC
GTCTTTAGGTTGGGGGGAGGGGTTTTATGCGATGGAGTTTCCCCACACTGAGTGG
GTGGAGACTGAAGTTAGGCCAGCTTGGCACTTGATGTAATTCTCCTTGGAATTTG
CCCTTTTTGAGTTTGGATCTTGGTTCATTCTCAAGCCTCAGACAGTGGTTCAAAGT
TTTTTTCTTCCATTTCAGGTGTCGTGA
SEQ ID NO: 7 EF1S
CGGCTCCGGTGCCCGTCAGTGGGCAGAGCGCACATCGCCCACAGTCCCCGAGAA
GTTGGGGGGAGGGGTCGGCAATTGAACCGGTGCCTAGAGAAGGTGGCGCGGGGT
AAACTGGGAAAGTGATGTCGTGTACTGGCTCCGCCTTTTTCCCGAGGGTGGGGGA
GAACCGTATATAAGTGCAGTAGTCGCCGTGAACGTTCTTTTTCGCAACGGGTTTG
CCGCCAGAACACAG
SEQ ID NO: 8 hAAT S
AATGACTCCTTTCGGTAAGTGCAGTGGAAGCTGTACACTGCCCAGGCAAAGCGTC
CGGGCAGCGTAGGCGGGCGACTCAGATCCCAGCCAGTGGACTTAGCCCCTGTTT
GCTCCTCCGATAACTGGGGTGACCTTGGTTAATATTCACCAGCAGCCTCCCCCGT
TGCCCCTCTGGATCCACTGCTTAAATACGGACGAGGACAGGGCCCTGTCTCCTCA
GCTTCAGGCACCACCACTGACCTGGGACAGTG
SEQ ID NO: 9 hAAT L
TGCTACCAGTGGAACAGCCACTAAGGATTCTGCAGTGAGAGCAGAGGGCCAGCT
AAGTGGTACTCTCCCAGAGACTGTCTGACTCACGCCACCCCCTCCACCTTGGACA
CAGGACGCTGTGGTTTCTGAGCCAGGTACAATGACTCCTTTCGGTAAGTGCAGTG
GAAGCTGTACACTGCCCAGGCAAAGCGTCCGGGCAGCGTAGGCGGGCGACTCAG
ATCCCAGCCAGTGGACTTAGCCCCTGTTTGCTCCTCCGATAACTGGGGTGACCTT
GGTTAATATTCACCAGCAGCCTCCCCCGTTGCCCCTCTGGATCCACTGCTTAAAT
ACGGACGAGGACAGGGCCCTGTCTCCTCAGCTTCAGGCACCACCACTGACCTGG
GACAGTGAAT
SEQ ID NO: 10 APOE S
CCTAAAATGGGCAAACATTGCAAGCAGCAAACAGCAAACACACAGCCCTCCCTG
CCTGCTGACCTTGGAGCTGGGGCAGAGGTCAGAGACCTCTCTGGGCCCATGCCAC
CTCCAACATCCACTCGACCCCTTGGAATTTCGGTGGAGAGGAGCAGAGGTTGTCC
TGGCGTGGTTTAGGTAGTGTGAGAG
SEQ ID NO: 11 APOE L
AGGCTCAGAGGCACACAGGAGTTTCTGGGCTCACCCTGCCCCCTTCCAACCCCTC
AGTTCCCATCCTCCAGCAGCTGTTTGTGTGCTGCCTCTGAAGTCCACACTGAACA
AACTTCAGCCTACTCATGTCCCTAAAATGGGCAAACATTGCAAGCAGCAAACAG
CAAACACACAGCCCTCCCTGCCTGCTGACCTTGGAGCTGGGGCAGAGGTCAGAG
ACCTCTCTGGGCCCATGCCACCTCCAACATCCACTCGACCCCTTGGAATTTCGGT
GGAGAGGAGCAGAGGTTGTCCTGGCGTGGTTTAGGTAGTGTGAGA
SEQ ID NO: 12 Intron C
GTAAGTATCAAGGTTACAAGACAGGTTTAAGGAGACCAATAGAAACTGGGCTTG
TCGAGACAGAGAAGACTCTTGCGTTTCTGATAGGCACCTATTGGTCTTACTGACA
TCCACTTTGCCTTTCTCTCCACAG
SEQ ID NO: 13 Intron B
GTACACATATTGACCAAATCAGGGTAATTTTGCATTTGTAATTTTAAAAAATGCT
TTCTTCTTTTAATATACTTTTTTGTTTATCTTATTTCTAATACTTTCCCTAATCTCTT
TCTTTCAGGGCAATAATGATACAATGTATCATGCCTCTTTGCACCATTCTAAAGA
ATAACAGTGATAATTTCTGGGTTAAGGCAATAGCAATATTTCTGCATATAAATAT
TTCTGCATATAAATTGTAACTGATGTAAGAGGTTTCATATTGCTAATAGCAGCTA
CAATCCAGCTACCATTCTGCTTTTATTTTCTGGTTGGGATAAGGCTGGATTATTCT
GAGTCCAAGCTAGGCCCTTTTGCTAATCTTGTTCATACCTCTTATCTTCCTCCCAC
AGCTCCTGGGCAACCTGCTGGTCTCTCTGCTGGCCCATCACTTTGGCAAAGGAAT
TC
SEQ ID NO: 14 Intron S
GTCGATCCTACCATCCACTCGACACACCCGCCAGCGGCCGCGTTGGTATCAAGGT
TACAAGACAGGTTTAAGGAGACCAATAGAAACTGGGCATGTGGAGACAGAGAA
GACTCTTGGGTTTCTGATAGGCACTGACTCTCTTCCTTTGTCCTGTTCCCATTTCA
G
SEQ ID NO: 15 HPRE
ATAACAGGCCTATTGATTGGAAAGTTTGTCAACGAATTGTGGGTCTTTTGGGGTT
TGCTGCCCCTTTTACGCAATGTGGATATCCTGCTTTAATGCCTTTATATGCATGTA
TACAAGCAAAACAGGCTTTTACTTTCTCGCCAACTTACAAGGCCTTTCTCAGTAA
ACAGTATATGACCCTTTACCCCGTTGCTCGGCAACGGCCTGGTCTGTGCCAAGTG
TTTGCTGACGCAACCCCCACTGGTTGGGGCTTGGCCATAGGCCATCAGCGCATGC
GTGGAACCTTTGTGTCTCCTCTGCCGATCCATACTGCGGAACTCCTAGCCGCTTGT
TTTGCTCGCAGCAGGTCTGGAGCAAACCTCATCGGGACCGACAATTCTGTCGTAC
TCTCCCGCAAGTATACATCGTTTCCATGGCTGCTAGGCTGTGCTGCCAACTGGAT
CCTGCGCGGGACGTCCTTTGTTTACGTCCCGTCGGCGCTGAATCCCGCGGACGAC
CCCTCCCGGGGCCGCTTGGGGCTCTACCGCCCGCTTCTCCGTCTGCCGTACCGTCC
GACCACGGGGCGCACCTCTCTTTACGCGGACTCCCCGTCTGTGCCTTCTCATCTGC
CGGACCGTGTGCACTTCGCTTCACCTCTGCACGTCGCATGGAGGCCACCGTGAAC
GCCCACCGGAACCTGCCCAAGGTCTTGCATAAGAGGACTCTTGGACTTTCAGCAA
TGTCATC
SEQ ID NO: 16 Bovine poly A
GATCTTTTTCCCTCTGCCAAAAATTATGGGGACATCATGAAGCCCCTTGAGCATC
TGACTTCTGGCTAATAAAGGAAATTTATTTTCATTGCAATAGTGTGTTGGAATTTT
TTGTGTCTCTCACTCG
SEQ ID NO: 17 Rabbit poly A
GATCTTTTTCCCTCTGCCAAAAATTATGGGGACATCATGAAGCCCCTTGAGCATC
TGACTTCTGGCTAATAAAGGAAATTTATTTTCATTGCAATAGTGTGTTGGAATTTT
TTGTGTCTCTCACTCG
SEQ ID NO: 18 pAAV EF1L intron C synMMUT1 kana
CTTCCGCTTCCTCGCTCACTGACTCGCTGCGCTCGGTCGTTCGGCTGCGGCGAGC
GGTATCAGCTCACTCAAAGGCGGTAATACGGTTATCCACAGAATCAGGGGATAA
CGCAGGAAAGAACATGTGAGCAAAAGGCCAGCAAAAGGCCAGGAACCGTAAAA
AGGCCGCGTTGCTGGCGTTTTTCCATAGGCTCCGCCCCCCTGACGAGCATCACAA
AAATCGACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGCCGCTTA
CCGGATACCTGTCCGCCTTTCTCCCTTCGGGAAGCGTGGCGCTTTCTCATAGCTCA
CGCTGTAGGTATCTCAGTTCGGTGTAGGTCGTTCGCTCCAAGCTGGGCTGTGTGC
ACGAACCCCCCGTTCAGCCCGACCGCTGCGCCTTATCCGGTAACTATCGTCTTGA
GTCCAACCCGGTAAGACACGACTTATCGCCACTGGCAGCAGCCACTGGTAACAG
GATTAGCAGAGCGAGGTATGTAGGCGGTGCTACAGAGTTCTTGAAGTGGTGGCC
TAACTACGGCTACACTAGAAGAACAGTATTTGGTATCTGCGCTCTGCTGAAGCCA
GTTACCTTCGGAAAAAGAGTTGGTAGCTCTTGATCCGGCAAACAAACCACCGCTG
GTAGCGGTGGTTTTTTTGTTTGCAAGCAGCAGATTACGCGCAGAAAAAAAGGATC
TCAAGAAGATCCTTTGATCTTTTCTACGGGGTCTGACGCTCAGTGGAACGAAAAC
TCACGTTAAGGGATTTTGGTCATGAGATTATCAAAAAGGATCTTCACCTAGATCC
TTTTAAATTAAAAATGAAGTTTTAAATCAATCTAAAGTATATATGAGTAAACTTG
GTCTGACAGTTAGAAAAACTCATCGAGCATCAAATGAAACTGCAATTTATTCATA
TCAGGATTATCAATACCATATTTTTGAAAAAGCCGTTTCTGTAATGAAGGAGAAA
ACTCACCGAGGCAGTTCCATAGGATGGCAAGATCCTGGTATCGGTCTGCGATTCC
GACTCGTCCAACATCAATACAACCTATTAATTTCCCCTCGTCAAAAATAAGGTTA
TCAAGTGAGAAATCACCATGAGTGACGACTGAATCCGGTGAGAATGGCAAAAGT
TTATGCATTTCTTTCCAGACTTGTTCAACAGGCCAGCCATTACGCTCGTCATCAAA
ATCACTCGCATCAACCAAACCGTTATTCATTCGTGATTGCGCCTGAGCGAGGCGA
AATACGCGATCGCTGTTAAAAGGACAATTACAAACAGGAATCGAGTGCAACCGG
CGCAGGAACACTGCCAGCGCATCAACAATATTTTCACCTGAATCAGGATATTCTT
CTAATACCTGGAACGCTGTTTTTCCGGGGATCGCAGTGGTGAGTAACCATGCATC
ATCAGGAGTACGGATAAAATGCTTGATGGTCGGAAGTGGCATAAATTCCGTCAG
CCAGTTTAGTCTGACCATCTCATCTGTAACATCATTGGCAACGCTACCTTTGCCAT
GTTTCAGAAACAACTCTGGCGCATCGGGCTTCCCATACAAGCGATAGATTGTCGC
ACCTGATTGCCCGACATTATCGCGAGCCCATTTATACCCATATAAATCAGCATCC
ATGTTGGAATTTAATCGCGGCCTCGACGTTTCCCGTTGAATATGGCTCATACTCTT
CCTTTTTCAATATTATTGAAGCATTTATCAGGGTTATTGTCTCATGAGCGGATACA
TATTTGAATGTATTTAGAAAAATAAACAAATAGGGGTTCCGCGCACATTTCCCCG
AAAAGTGCCACCTGACGTCTAAGAAACCATTATTATCATGACATTAACCTATAAA
AATAGGCGTATCACGAGGCCCTTTCGTCTCGCGCGTTTCGGTGATGACGGTGAAA
ACCTCTGACACATGCAGCTCCCGGAGACGGTCACAGCTTGTCTGTAAGCGGATGC
CGGGAGCAGACAAGCCCGTCAGGGCGCGTCAGCGGGTGTTGGCGGGTGTCGGGG
CTGGCTTAACTATGCGGCATCAGAGCAGATTGTACTGAGAGTGCACCATTCGACG
CTCTCCCTTATGCGACTCCTGCATTAGGAAGCAGCCCAGTAGTAGGTTGAGGCCG
TTGAGCACCGCCGCCGCAAGGAATGGTGCATGCAAGGAGATGGCGCCCAACAGT
CCCCCGGCCACGGGGCCTGCCACCATACCCACGCCGAAACAAGCGCTCATGAGC
CCGAAGTGGCGAGCCCGATCTTCCCCATCGGTGATGTCGGCGATATAGGCGCCA
GCAACCGCACCTGTGGCGCCGGTGATGCCGGCCACGATGCGTCCGGCGTAGAGG
ATCTGGCTAGCGATGACCCTGCTGATTGGTTCGCTGACCATTTCCGGGTGCGGGA
CGGCGTTACCAGAAACTCAGAAGGTTCGTCCAACCAAACCGACTCTGACGGCAG
TTTACGAGAGAGATGATAGGGTCTGCTTCAGTAAGCCAGATGCTACACAATTAG
GCTTGTACATATTGTCGTTAGAACGCGGCTACAATTAATACATAACCTTATGTAT
CATACACATACGATTTAGGTGACACTATAGAATACACGGAATTAATTCTTGGCCA
CTCCCTCTCTGCGCGCTCGCTCGCTCACTGAGGCCGCCCGGGCAAAGCCCGGGCG
TCGGGCGACCTTTGGTCGCCCGGCCTCAGTGAGCGAGCGAGCGCGCAGAGAGGG
AGTGGCCAACTCCATCACTAGGGGTTCCTTACGTAGCCATGCTCTAGCGATCGCG
GTACCTCTAGAGGCTCCGGTGCCCGTCAGTGGGCAGAGCGCACATCGCCCACAG
TCCCCGAGAAGTTGGGGGGAGGGGTCGGCAATTGAACCGGTGCCTAGAGAAGGT
GGCGCGGGGTAAACTGGGAAAGTGATGTCGTGTACTGGCTCCGCCTTTTTCCCGA
GGGTGGGGGAGAACCGTATATAAGTGCAGTAGTCGCCGTGAACGTTCTTTTTCGC
AACGGGTTTGCCGCCAGAACACAGGTAAGTGCCGTGTGTGGTTCCCGCGGGCCT
GGCCTCTTTACGGGTTATGGCCCTTGCGTGCCTTGAATTACTTCCACCTGGCTGCA
GTACGTGATTCTTGATCCCGAGCTTCGGGTTGGAAGTGGGTGGGAGAGTTCGAGG
CCTTGCGCTTAAGGAGCCCCTTCGCCTCGTGCTTGAGTTGAGGCCTGGCCTGGGC
GCTGGGGCCGCCGCGTGCGAATCTGGTGGCACCTTCGCGCCTGTCTCGCTGCTTT
CGATAAGTCTCTAGCCATTTAAAATTTTTGATGACCTGCTGCGACGCTTTTTTTCT
GGCAAGATAGTCTTGTAAATGCGGGCCAAGATCTGCACACTGGTATTTCGGTTTT
TGGGGCCGCGGGCGGCGACGGGGCCCGTGCGTCCCAGCGCACATGTTCGGCGAG
GCGGGGCCTGCGAGCGCGGCCACCGAGAATCGGACGGGGGTAGTCTCAAGCTGG
CCGGCCTGCTCTGGTGCCTGGCCTCGCGCCGCCGTGTATCGCCCCGCCCTGGGCG
GCAAGGCTGGCCCGGTCGGCACCAGTTGCGTGAGCGGAAAGATGGCCGCTTCCC
GGCCCTGCTGCAGGGAGCTCAAAATGGAGGACGCGGCGCTCGGGAGAGCGGGC
GGGTGAGTCACCCACACAAAGGAAAAGGGCCTTTCCGTCCTCAGCCGTCGCTTCA
TGTGACTCCACGGAGTACCGGGCGCCGTCCAGGCACCTCGATTAGTTCTCGAGCT
TTTGGAGTACGTCGTCTTTAGGTTGGGGGGAGGGGTTTTATGCGATGGAGTTTCC
CCACACTGAGTGGGTGGAGACTGAAGTTAGGCCAGCTTGGCACTTGATGTAATTC
TCCTTGGAATTTGCCCTTTTTGAGTTTGGATCTTGGTTCATTCTCAAGCCTCAGAC
AGTGGTTCAAAGTTTTTTTCTTCCATTTCAGGTGTCGTGAGCTAGAGCTTTATTGC
GGTAGTTTATCACAGTTAAATTGCTAACGCAGTCAGTGCTTCTGACACAACAGTC
TCGAACTTAAGCTGCAGAAGTTGGTCGTGAGGCACTGGGCAGGTAAGTATCAAG
GTTACAAGACAGGTTTAAGGAGACCAATAGAAACTGGGCTTGTCGAGACAGAGA
AGACTCTTGCGTTTCTGATAGGCACCTATTGGTCTTACTGACATCCACTTTGCCTT
TCTCTCCACAGGTGTCCACTCCCAGTTCAATTACAGCTCTTAAGGCTAGAGTACTT
AATACGACTCACTATAGGCTAGCGCGTCCACCATGCTGAGAGCCAAAAACCAGC
TGTTCCTGCTGAGCCCCCACTATCTGAGACAGGTCAAAGAAAGTTCCGGGAGTAG
ACTGATCCAGCAGAGACTGCTGCACCAGCAGCAGCCACTGCATCCTGAGTGGGC
CGCTCTGGCCAAGAAACAGCTGAAGGGCAAAAACCCAGAAGACCTGATCTGGCA
CACTCCAGAGGGGATTTCAATCAAGCCCCTGTACAGCAAAAGGGACACTATGGA
TCTGCCAGAGGAACTGCCAGGAGTGAAGCCTTTCACCCGCGGACCTTACCCAACT
ATGTATACCTTTCGACCCTGGACAATTCGGCAGTACGCCGGCTTCAGTACTGTGG
AGGAATCAAACAAGTTTTATAAGGACAACATCAAGGCTGGACAGCAGGGCCTGA
GTGTGGCATTCGATCTGGCCACACATCGCGGCTATGACTCAGATAATCCCAGAGT
CAGGGGGGACGTGGGAATGGCAGGAGTCGCTATCGACACAGTGGAAGATACTAA
GATTCTGTTCGATGGAATCCCTCTGGAGAAAATGTCTGTGAGTATGACAATGAAC
GGCGCTGTCATTCCCGTGCTGGCAAACTTCATCGTCACTGGCGAGGAACAGGGG
GTGCCTAAGGAAAAACTGACCGGCACAATTCAGAACGACATCCTGAAGGAGTTC
ATGGTGCGGAATACTTACATTTTTCCCCCTGAACCATCCATGAAAATCATTGCCG
ATATCTTCGAGTACACCGCTAAGCACATGCCCAAGTTCAACTCAATTAGCATCTC
CGGGTATCATATGCAGGAAGCAGGAGCCGACGCTATTCTGGAGCTGGCTTACAC
CCTGGCAGATGGCCTGGAATATTCTCGAACCGGACTGCAGGCAGGCCTGACAAT
CGACGAGTTCGCTCCTAGACTGAGTTTCTTTTGGGGAATTGGCATGAACTTTTAC
ATGGAGATCGCCAAGATGAGGGCTGGCCGGAGACTGTGGGCACACCTGATCGAG
AAGATGTTCCAGCCTAAGAACTCTAAGAGTCTGCTGCTGCGGGCCCATTGCCAGA
CATCCGGCTGGTCTCTGACTGAACAGGACCCATATAACAATATTGTCAGAACCGC
AATCGAGGCAATGGCAGCCGTGTTCGGAGGAACCCAGAGCCTGCACACAAACTC
CTTTGATGAGGCCCTGGGGCTGCCTACCGTGAAGTCTGCTAGGATTGCACGCAAT
ACACAGATCATTATCCAGGAGGAATCCGGAATCCCAAAGGTGGCCGATCCCTGG
GGAGGCTCTTACATGATGGAGTGCCTGACAAACGACGTGTATGATGCTGCACTG
AAGCTGATTAATGAAATCGAGGAAATGGGGGGAATGGCAAAGGCCGTGGCTGA
GGGCATTCCAAAACTGAGGATCGAGGAATGTGCAGCTAGGCGCCAGGCACGAAT
TGACTCAGGAAGCGAAGTGATCGTCGGGGTGAATAAGTACCAGCTGGAGAAAGA
AGACGCAGTCGAAGTGCTGGCCATCGATAACACAAGCGTGCGCAATCGACAGAT
TGAGAAGCTGAAGAAAATCAAAAGCTCCCGCGATCAGGCACTGGCCGAACGATG
CCTGGCAGCCCTGACTGAGTGTGCTGCAAGCGGGGACGGAAACATTCTGGCTCT
GGCAGTCGATGCCTCCCGGGCTAGATGCACTGTGGGGGAAATCACCGACGCCCT
GAAGAAAGTCTTCGGAGAGCACAAGGCCAATGATCGGATGGTGAGCGGCGCTTA
TAGACAGGAGTTCGGGGAATCTAAAGAGATTACCAGTGCCATCAAGAGGGTGCA
CAAGTTCATGGAGAGAGAAGGGCGACGGCCCAGGCTGCTGGTGGCAAAGATGG
GACAGGACGGACATGATCGCGGAGCAAAAGTCATTGCCACCGGGTTCGCTGACC
TGGGATTTGACGTGGATATCGGCCCTCTGTTCCAGACACCACGAGAGGTCGCACA
GCAGGCAGTCGACGCTGATGTGCACGCAGTCGGAGTGTCCACTCTGGCAGCTGG
CCATAAGACCCTGGTGCCTGAACTGATCAAAGAGCTGAACTCTCTGGGCAGACC
AGACATCCTGGTCATGTGCGGCGGCGTGATCCCACCCCAGGATTACGAATTCCTG
TTTGAGGTCGGGGTGAGCAACGTGTTCGGACCAGGAACCAGGATCCCTAAGGCC
GCAGTGCAGGTCCTGGATGATATTGAAAAGTGTCTGGAAAAGAAACAGCAGTCA
GTGTAACTCGAGGACGGGGTGAACTACGCCTGAGGATCCGATCTTTTTCCCTCTG
CCAAAAATTATGGGGACATCATGAAGCCCCTTGAGCATCTGACTTCTGGCTAATA
AAGGAAATTTATTTTCATTGCAATAGTGTGTTGGAATTTTTTGTGTCTCTCACTCG
TCTAGAACTAGTCCTGCAGGTAGAGCATGGCTACGTAAGGAACCCCTAGTGATG
GAGTTGGCCACTCCCTCTCTGCGCGCTCGCTCGCTCACTGAGGCCGGGCGACCAA
AGGTCGCCCGACGCCCGGGCTTTGCCCGGGCGGCCTCAGTGAGCGAGCGAGCGC
GCAGAGAGGGAGTGGCCAACTTTTTGCAAAAGCCTAGGCCTCCAAAAAAGCCTC
CTCACTACTTCTGGAATAGCTCAGAGGCCGAGGCGGCCTCGGCCTCTGCATAAAT
AAAAAAAATTAGTCAGCCATGGGGCGGAGAATGGGCGGAACTGGGCGGAGTTA
GGGGCGGGATGGGCGGAGTTAGGGGCGGGACTATGGTTGCTGACTAATTGAGAT
GCATGCTTTGCATACTTCTGCCTGCTGGGGAGCCTGGGGACTTTCCACACCTGGT
TGCTGACTAATTGAGATGCATGCTTTGCATACTTCTGCCTGCTGGGGAGCCTGGG
GACTTTCCACACCCTAACTGACACACATTCCACAGCTG
SEQ ID NO: 19 pAAV hAAT synMMUT1 Amp
GCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGC
CAACTCCATCACTAGGGGTTCCTTGTAGTTAATGATTAACCCGCCATGCTACTTAT
CTACCAGGGTAATGGGGATCCTCTAGAACTATAGCTAGTCGACATTGATTATTGA
CTAGTCCTAAAATGGGCAAACATTGCAAGCAGCAAACAGCAAACACACAGCCCT
CCCTGCCTGCTGACCTTGGAGCTGGGGCAGAGGTCAGAGACCTCTCTGGGCCCAT
GCCACCTCCAACATCCACTCGACCCCTTGGAATTTCGGTGGAGAGGAGCAGAGG
TTGTCCTGGCGTGGTTTAGGTAGTGTGAGAGGGCGCGCCAATGACTCCTTTCGGT
AAGTGCAGTGGAAGCTGTACACTGCCCAGGCAAAGCGTCCGGGCAGCGTAGGCG
GGCGACTCAGATCCCAGCCAGTGGACTTAGCCCCTGTTTGCTCCTCCGATAACTG
GGGTGACCTTGGTTAATATTCACCAGCAGCCTCCCCCGTTGCCCCTCTGGATCCA
CTGCTTAAATACGGACGAGGACAGGGCCCTGTCTCCTCAGCTTCAGGCACCACCA
CTGACCTGGGACAGTGACGCGTCCACCATGCTGAGAGCCAAAAACCAGCTGTTC
CTGCTGAGCCCCCACTATCTGAGACAGGTCAAAGAAAGTTCCGGGAGTAGACTG
ATCCAGCAGAGACTGCTGCACCAGCAGCAGCCACTGCATCCTGAGTGGGCCGCT
CTGGCCAAGAAACAGCTGAAGGGCAAAAACCCAGAAGACCTGATCTGGCACACT
CCAGAGGGGATTTCAATCAAGCCCCTGTACAGCAAAAGGGACACTATGGATCTG
CCAGAGGAACTGCCAGGAGTGAAGCCTTTCACCCGCGGACCTTACCCAACTATGT
ATACCTTTCGACCCTGGACAATTCGGCAGTACGCCGGCTTCAGTACTGTGGAGGA
ATCAAACAAGTTTTATAAGGACAACATCAAGGCTGGACAGCAGGGCCTGAGTGT
GGCATTCGATCTGGCCACACATCGCGGCTATGACTCAGATAATCCCAGAGTCAGG
GGGGACGTGGGAATGGCAGGAGTCGCTATCGACACAGTGGAAGATACTAAGATT
CTGTTCGATGGAATCCCTCTGGAGAAAATGTCTGTGAGTATGACAATGAACGGCG
CTGTCATTCCCGTGCTGGCAAACTTCATCGTCACTGGCGAGGAACAGGGGGTGCC
TAAGGAAAAACTGACCGGCACAATTCAGAACGACATCCTGAAGGAGTTCATGGT
GCGGAATACTTACATTTTTCCCCCTGAACCATCCATGAAAATCATTGCCGATATC
TTCGAGTACACCGCTAAGCACATGCCCAAGTTCAACTCAATTAGCATCTCCGGGT
ATCATATGCAGGAAGCAGGAGCCGACGCTATTCTGGAGCTGGCTTACACCCTGG
CAGATGGCCTGGAATATTCTCGAACCGGACTGCAGGCAGGCCTGACAATCGACG
AGTTCGCTCCTAGACTGAGTTTCTTTTGGGGAATTGGCATGAACTTTTACATGGA
GATCGCCAAGATGAGGGCTGGCCGGAGACTGTGGGCACACCTGATCGAGAAGAT
GTTCCAGCCTAAGAACTCTAAGAGTCTGCTGCTGCGGGCCCATTGCCAGACATCC
GGCTGGTCTCTGACTGAACAGGACCCATATAACAATATTGTCAGAACCGCAATCG
AGGCAATGGCAGCCGTGTTCGGAGGAACCCAGAGCCTGCACACAAACTCCTTTG
ATGAGGCCCTGGGGCTGCCTACCGTGAAGTCTGCTAGGATTGCACGCAATACAC
AGATCATTATCCAGGAGGAATCCGGAATCCCAAAGGTGGCCGATCCCTGGGGAG
GCTCTTACATGATGGAGTGCCTGACAAACGACGTGTATGATGCTGCACTGAAGCT
GATTAATGAAATCGAGGAAATGGGGGGAATGGCAAAGGCCGTGGCTGAGGGCA
TTCCAAAACTGAGGATCGAGGAATGTGCAGCTAGGCGCCAGGCACGAATTGACT
CAGGAAGCGAAGTGATCGTCGGGGTGAATAAGTACCAGCTGGAGAAAGAAGAC
GCAGTCGAAGTGCTGGCCATCGATAACACAAGCGTGCGCAATCGACAGATTGAG
AAGCTGAAGAAAATCAAAAGCTCCCGCGATCAGGCACTGGCCGAACGATGCCTG
GCAGCCCTGACTGAGTGTGCTGCAAGCGGGGACGGAAACATTCTGGCTCTGGCA
GTCGATGCCTCCCGGGCTAGATGCACTGTGGGGGAAATCACCGACGCCCTGAAG
AAAGTCTTCGGAGAGCACAAGGCCAATGATCGGATGGTGAGCGGCGCTTATAGA
CAGGAGTTCGGGGAATCTAAAGAGATTACCAGTGCCATCAAGAGGGTGCACAAG
TTCATGGAGAGAGAAGGGCGACGGCCCAGGCTGCTGGTGGCAAAGATGGGACA
GGACGGACATGATCGCGGAGCAAAAGTCATTGCCACCGGGTTCGCTGACCTGGG
ATTTGACGTGGATATCGGCCCTCTGTTCCAGACACCACGAGAGGTCGCACAGCAG
GCAGTCGACGCTGATGTGCACGCAGTCGGAGTGTCCACTCTGGCAGCTGGCCATA
AGACCCTGGTGCCTGAACTGATCAAAGAGCTGAACTCTCTGGGCAGACCAGACA
TCCTGGTCATGTGCGGCGGCGTGATCCCACCCCAGGATTACGAATTCCTGTTTGA
GGTCGGGGTGAGCAACGTGTTCGGACCAGGAACCAGGATCCCTAAGGCCGCAGT
GCAGGTCCTGGATGATATTGAAAAGTGTCTGGAAAAGAAACAGCAGTCAGTGTA
ACTCGAGGACGGGGTGAACTACGCCTGAGGATCCGATCTTTTTCCCTCTGCCAAA
AATTATGGGGACATCATGAAGCCCCTTGAGCATCTGACTTCTGGCTAATAAAGGA
AATTTATTTTCATTGCAATAGTGTGTTGGAATTTTTTGTGTCTCTCACTCGGAAGC
AATTCGTTGATCTGAATTTCGACCACCCATAATACCCATTACCCTGGTAGATAAG
TAGCATGGCGGGTTAATCATTAACTACAAGGAACCCCTAGTGATGGAGTTGGCC
ACTCCCTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAGCCTTA
ATTAACCTAATTCACTGGCCGTCGTTTTACAACGTCGTGACTGGGAAAACCCTGG
CGTTACCCAACTTAATCGCCTTGCAGCACATCCCCCTTTCGCCAGCTGGCGTAAT
AGCGAAGAGGCCCGCACCGATCGCCCTTCCCAACAGTTGCGCAGCCTGAATGGC
GAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTT
CCCTTCCTTTCTCGCCACGTTCGCCGGCTTTCCCCGTCAAGCTCTAAATCGGGGGC
TCCCTTTAGGGTTCCGATTTAGTGCTTTACGGCACCTCGACCCCAAAAAACTTGA
TTAGGGTGATGGTTCACGTAGTGGGCCATCGCCCTGATAGACGGTTTTTCGCCCT
TTGACGTTGGAGTCCACGTTCTTTAATAGTGGACTCTTGTTCCAAACTGGAACAA
CACTCAACCCTATCTCGGTCTATTCTTTTGATTTATAAGGGATTTTGCCGATTTCG
GCCTATTGGTTAAAAAATGAGCTGATTTAACAAAAATTTAACGCGAATTTTAACA
AAATATTAACGCTTACAATTTAGGTGGCACTTTTCGGGGAAATGTGCGCGGAACC
CCTATTTGTTTATTTTTCTAAATACATTCAAATATGTATCCGCTCATGAGACAATA
ACCCTGATAAATGCTTCAATAATATTGAAAAAGGAAGAGTATGAGTATTCAACA
TTTCCGTGTCGCCCTTATTCCCTTTTTTGCGGCATTTTGCCTTCCTGTTTTTGCTCA
CCCAGAAACGCTGGTGAAAGTAAAAGATGCTGAAGATCAGTTGGGTGCACGAGT
GGGTTACATCGAACTGGATCTCAACAGCGGTAAGATCCTTGAGAGTTTTCGCCCC
GAAGAACGTTTTCCAATGATGAGCACTTTTAAAGTTCTGCTATGTGGCGCGGTAT
TATCCCGTATTGACGCCGGGCAAGAGCAACTCGGTCGCCGCATACACTATTCTCA
GAATGACTTGGTTGAGTACTCACCAGTCACAGAAAAGCATCTTACGGATGGCAT
GACAGTAAGAGAATTATGCAGTGCTGCCATAACCATGAGTGATAACACTGCGGC
CAACTTACTTCTGACAACGATCGGAGGACCGAAGGAGCTAACCGCTTTTTTGCAC
AACATGGGGGATCATGTAACTCGCCTTGATCGTTGGGAACCGGAGCTGAATGAA
GCCATACCAAACGACGAGCGTGACACCACGATGCCTGTAGCAATGGCAACAACG
TTGCGCAAACTATTAACTGGCGAACTACTTACTCTAGCTTCCCGGCAACAATTAA
TAGACTGGATGGAGGCGGATAAAGTTGCAGGACCACTTCTGCGCTCGGCCCTTCC
GGCTGGCTGGTTTATTGCTGATAAATCTGGAGCCGGTGAGCGTGGGTCTCGCGGT
ATCATTGCAGCACTGGGGCCAGATGGTAAGCCCTCCCGTATCGTAGTTATCTACA
CGACGGGGAGTCAGGCAACTATGGATGAACGAAATAGACAGATCGCTGAGATAG
GTGCCTCACTGATTAAGCATTGGTAACTGTCAGACCAAGTTTACTCATATATACT
TTAGATTGATTTAAAACTTCATTTTTAATTTAAAAGGATCTAGGTGAAGATCCTTT
TTGATAATCTCATGACCAAAATCCCTTAACGTGAGTTTTCGTTCCACTGAGCGTC
AGACCCCGTAGAAAAGATCAAAGGATCTTCTTGAGATCCTTTTTTTCTGCGCGTA
ATCTGCTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCGCAGAT
ACCAAATACTGTTCTTCTAGTGTAGCCGTAGTTAGGCCACCACTTCAAGAACTCT
GTAGCACCGCCTACATACCTCGCTCTGCTAATCCTGTTACCAGTGGCTGCTGCCA
GTGGCGATAAGTCGTGTCTTACCGGGTTGGACTCAAGACGATAGTTACCGGATAA
GGCGCAGCGGTCGGGCTGAACGGGGGGTTCGTGCACACAGCCCAGCTTGGAGCG
AACGACCTACACCGAACTGAGATACCTACAGCGTGAGCTATGAGAAAGCGCCAC
GCTTCCCGAAGGGAGAAAGGCGGACAGGTATCCGGTAAGCGGCAGGGTCGGAA
CAGGAGAGCGCACGAGGGAGCTTCCAGGGGGAAACGCCTGGTATCTTTATAGTC
CTGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGCCT
TTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGA
GCGCAGCGAGTCAGTGAGCGAGGAAGCGGAAGAGCGCCCAATACGCAAACCGC
CTCTCCCCGCGCGTTGGCCGATTCATTAATGCAGCTGGCACGACAGGTTTCCCGA
CTGGAAAGCGGGCAGTGAGCGCAACGCAATTAATGTGAGTTAGCTCACTCATTA
GGCACCCCAGGCTTTACACTTTATGCTTCCGGCTCGTATGTTGTGTGGAATTGTGA
GCGGATAACAATTTCACACAGGAAACAGCTATGACCATGATTACGCCAGATTTA
ATTAAGGCCTTAATTAG
SEQ ID NO: 20 pAAV ITR 130 EF1S synMMUT1 HPRE kana
CCTGCAGGCAGCTGCGCGCTCGCTCGCTCACTGAGGCCGCCCGGGCGTCGGGCG
ACCTTTGGTCGCCCGGCCTCAGTGAGCGAGCGAGCGCGCAGAGAGGGAGTGGCC
AACTCCATCACTAGGGGTTCCTGCGGCCGTAGGTACCGGCTCCGGTGCCCGTCAG
TGGGCAGAGCGCACATCGCCCACAGTCCCCGAGAAGTTGGGGGGAGGGGTCGGC
AATTGAACCGGTGCCTAGAGAAGGTGGCGCGGGGTAAACTGGGAAAGTGATGTC
GTGTACTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAGGTCAG
ATCAGATCTTTGTCGATCCTACCATCCACTCGACACACCCGCCAGCGGCCGCGTT
GGTATCAAGGTTACAAGACAGGTTTAAGGAGACCAATAGAAACTGGGCATGTGG
AGACAGAGAAGACTCTTGGGTTTCTGATAGGCACTGACTCTCTTCCTTTGTCCTGT
TCCCATTTCAGAAGCTTCCGAGCTCTCGAATTCGAGCTCGGTACCTCGCGTGCAT
CTAGATAATCCACCATGCTGAGAGCCAAAAACCAGCTGTTCCTGCTGAGCCCCCA
CTATCTGAGACAGGTCAAAGAAAGTTCCGGGAGTAGACTGATCCAGCAGAGACT
GCTGCACCAGCAGCAGCCACTGCATCCTGAGTGGGCCGCTCTGGCCAAGAAACA
GCTGAAGGGCAAAAACCCAGAAGACCTGATCTGGCACACTCCAGAGGGGATTTC
AATCAAGCCCCTGTACAGCAAAAGGGACACTATGGATCTGCCAGAGGAACTGCC
AGGAGTGAAGCCTTTCACCCGCGGACCTTACCCAACTATGTATACCTTTCGACCC
TGGACAATTCGGCAGTACGCCGGCTTCAGTACTGTGGAGGAATCAAACAAGTTTT
ATAAGGACAACATCAAGGCTGGACAGCAGGGCCTGAGTGTGGCATTCGATCTGG
CCACACATCGCGGCTATGACTCAGATAATCCCAGAGTCAGGGGGGACGTGGGAA
TGGCAGGAGTCGCTATCGACACAGTGGAAGATACTAAGATTCTGTTCGATGGAA
TCCCTCTGGAGAAAATGTCTGTGAGTATGACAATGAACGGCGCTGTCATTCCCGT
GCTGGCAAACTTCATCGTCACTGGCGAGGAACAGGGGGTGCCTAAGGAAAAACT
GACCGGCACAATTCAGAACGACATCCTGAAGGAGTTCATGGTGCGGAATACTTA
CATTTTTCCCCCTGAACCATCCATGAAAATCATTGCCGATATCTTCGAGTACACC
GCTAAGCACATGCCCAAGTTCAACTCAATTAGCATCTCCGGGTATCATATGCAGG
AAGCAGGAGCCGACGCTATTCTGGAGCTGGCTTACACCCTGGCAGATGGCCTGG
AATATTCTCGAACCGGACTGCAGGCAGGCCTGACAATCGACGAGTTCGCTCCTAG
ACTGAGTTTCTTTTGGGGAATTGGCATGAACTTTTACATGGAGATCGCCAAGATG
AGGGCTGGCCGGAGACTGTGGGCACACCTGATCGAGAAGATGTTCCAGCCTAAG
AACTCTAAGAGTCTGCTGCTGCGGGCCCATTGCCAGACATCCGGCTGGTCTCTGA
CTGAACAGGACCCATATAACAATATTGTCAGAACCGCAATCGAGGCAATGGCAG
CCGTGTTCGGAGGAACCCAGAGCCTGCACACAAACTCCTTTGATGAGGCCCTGG
GGCTGCCTACCGTGAAGTCTGCTAGGATTGCACGCAATACACAGATCATTATCCA
GGAGGAATCCGGAATCCCAAAGGTGGCCGATCCCTGGGGAGGCTCTTACATGAT
GGAGTGCCTGACAAACGACGTGTATGATGCTGCACTGAAGCTGATTAATGAAAT
CGAGGAAATGGGGGGAATGGCAAAGGCCGTGGCTGAGGGCATTCCAAAACTGA
GGATCGAGGAATGTGCAGCTAGGCGCCAGGCACGAATTGACTCAGGAAGCGAAG
TGATCGTCGGGGTGAATAAGTACCAGCTGGAGAAAGAAGACGCAGTCGAAGTGC
TGGCCATCGATAACACAAGCGTGCGCAATCGACAGATTGAGAAGCTGAAGAAAA
TCAAAAGCTCCCGCGATCAGGCACTGGCCGAACGATGCCTGGCAGCCCTGACTG
AGTGTGCTGCAAGCGGGGACGGAAACATTCTGGCTCTGGCAGTCGATGCCTCCC
GGGCTAGATGCACTGTGGGGGAAATCACCGACGCCCTGAAGAAAGTCTTCGGAG
AGCACAAGGCCAATGATCGGATGGTGAGCGGCGCTTATAGACAGGAGTTCGGGG
AATCTAAAGAGATTACCAGTGCCATCAAGAGGGTGCACAAGTTCATGGAGAGAG
AAGGGCGACGGCCCAGGCTGCTGGTGGCAAAGATGGGACAGGACGGACATGAT
CGCGGAGCAAAAGTCATTGCCACCGGGTTCGCTGACCTGGGATTTGACGTGGAT
ATCGGCCCTCTGTTCCAGACACCACGAGAGGTCGCACAGCAGGCAGTCGACGCT
GATGTGCACGCAGTCGGAGTGTCCACTCTGGCAGCTGGCCATAAGACCCTGGTGC
CTGAACTGATCAAAGAGCTGAACTCTCTGGGCAGACCAGACATCCTGGTCATGTG
CGGCGGCGTGATCCCACCCCAGGATTACGAATTCCTGTTTGAGGTCGGGGTGAGC
AACGTGTTCGGACCAGGAACCAGGATCCCTAAGGCCGCAGTGCAGGTCCTGGAT
GATATTGAAAAGTGTCTGGAAAAGAAACAGCAGTCAGTGTAAAGACGCGTGGTA
CCTCTAGAGTCGACCCGGGCGGCCTCGAGATAACAGGCCTATTGATTGGAAAGTT
TGTCAACGAATTGTGGGTCTTTTGGGGTTTGCTGCCCCTTTTACGCAATGTGGATA
TCCTGCTTTAATGCCTTTATATGCATGTATACAAGCAAAACAGGCTTTTACTTTCT
CGCCAACTTACAAGGCCTTTCTCAGTAAACAGTATATGACCCTTTACCCCGTTGC
TCGGCAACGGCCTGGTCTGTGCCAAGTGTTTGCTGACGCAACCCCCACTGGTTGG
GGCTTGGCCATAGGCCATCAGCGCATGCGTGGAACCTTTGTGTCTCCTCTGCCGA
TCCATACTGCGGAACTCCTAGCCGCTTGTTTTGCTCGCAGCAGGTCTGGAGCAAA
CCTCATCGGGACCGACAATTCTGTCGTACTCTCCCGCAAGTATACATCGTTTCCAT
GGCTGCTAGGCTGTGCTGCCAACTGGATCCTGCGCGGGACGTCCTTTGTTTACGT
CCCGTCGGCGCTGAATCCCGCGGACGACCCCTCCCGGGGCCGCTTGGGGCTCTAC
CGCCCGCTTCTCCGTCTGCCGTACCGTCCGACCACGGGGCGCACCTCTCTTTACG
CGGACTCCCCGTCTGTGCCTTCTCATCTGCCGGACCGTGTGCACTTCGCTTCACCT
CTGCACGTCGCATGGAGGCCACCGTGAACGCCCACCGGAACCTGCCCAAGGTCT
TGCATAAGAGGACTCTTGGACTTTCAGCAATGTCATCGATATCGTCGACTCGCTG
ATCAGCCTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAAATGA
GGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGA
TGCGGTGGGCTCTATGGCTTCTGAGGCGGAAAGAACCAGCTGGGGCTCGACTAG
ACTAGTCCTGCAGGTACGGCCGCAGGAACCCCTAGTGATGGAGTTGGCCACTCCC
TCTCTGCGCGCTCGCTCGCTCACTGAGGCCGGGCGACCAAAGGTCGCCCGACGCC
CGGGCTTTGCCCGGGCGGCCTCAGTGAGCGAGCGAGCGCGCAGGGCGCCTGATG
CGGTATTTTCTCCTTACGCATCTGTGCGGTATTTCACACCGCATACGTCAAAGCA
ACCATAGTACGCGCCCTGTAGCGGCGCATTAAGCGCGGCGGGTGTGGTGGTTAC
GCGCAGCGTGACCGCTACACTTGCCAGCGCCTTAGCGCCCGCTCCTTTCGCTTTCT
TCCCTTCCTTTCTCGCCACGTTCGCCGGCTTTCCCCGTCAAGCTCTAAATCGGGGG
CTCCCTTTAGGGTTCCGATTTAGTGCTTTACGGCACCTCGACCCCAAAAAACTTG
ATTTGGGTGATGGTTCACGTAGTGGGCCATCGCCCTGATAGACGGTTTTTCGCCC
TTTGACGTTGGAGTCCACGTTCTTTAATAGTGGACTCTTGTTCCAAACTGGAACA
ACACTCAACTCTATCTCGGGCTATTCTTTTGATTTATAAGGGATTTTGCCGATTTC
GGTCTATTGGTTAAAAAATGAGCTGATTTAACAAAAATTTAACGCGAATTTTAAC
AAAATATTAACGTTTACAATTTTATGGTGCACTCTCAGTACAATCTGCTCTGATGC
CGCATAGTTAAGCCAGCCCCGACACCCGCCAACACCCGCTGACGCGCCCTGACG
GGCTTGTCTGCTCCCGGCATCCGCTTACAGACAAGCTGTGACCGTCTCCGGGAGC
TGCATGTGTCAGAGGTTTTCACCGTCATCACCGAAACGCGCGAGACGAAAGGGC
CTCGTGATACGCCTATTTTTATAGGTTAATGTCATGAACAATAAAACTGTCTGCTT
ACATAAACAGTAATACAAGGGGTGTTATGAGCCATATTCAACGGGAAACGTCGA
GGCCGCGATTAAATTCCAACATGGATGCTGATTTATATGGGTATAAATGGGCTCG
CGATAATGTCGGGCAATCAGGTGCGACAATCTATCGCTTGTATGGGAAGCCCGAT
GCGCCAGAGTTGTTTCTGAAACATGGCAAAGGTAGCGTTGCCAATGATGTTACAG
ATGAGACGGTCAGACTAAACTGGCTGACGGAATTTATGCCTCTTCCGACCATCAA
GCATTTTATCCGTACTCCTGATGATGCATGGTTACTCACCACTGCGATCCCTGGA
AAAACAGCATTCCAGGTATTAGAAGAATATCCTGATTCAGGTGAAAATATTGTTG
ATGCGCTGGCAGTGTTCCTGCGCCGGTTGCATTCGATTCCTGTTTGTAATTGTCCT
TTTAACAGCGATCGCGTATTTCGTCTCGCTCAGGCGCAATCACGAATGAATAACG
GTTTGGTTGATGCGAGTGATTTTGATGACGAGCGTAATGGCTGGCCTGTTGAACA
AGTCTGGAAAGAAATGCATAAACTTTTGCCATTCTCACCGGATTCAGTCGTCACT
CATGGTGATTTCTCACTTGATAACCTTATTTTTGACGAGGGGAAATTAATAGGTT
GTATTGATGTTGGACGAGTCGGAATCGCAGACCGATACCAGGATCTTGCCATCCT
ATGGAACTGCCTCGGTGAGTTTTCTCCTTCATTACAGAAACGGCTTTTTCAAAAA
TATGGTATTGATAATCCTGATATGAATAAATTGCAGTTTCATTTGATGCTCGATG
AGTTTTTCTAATCAGAATTGGTTAATTGGTTGTAACACTGGCAGAGCATTACGCT
GACTTGACGGGACGGCGCAAGCTCATGACCAAAATCCCTTAACGTGAGTTTTCGT
TCCACTGAGCGTCAGACCCCGTAGAAAAGATCAAAGGATCTTCTTGAGATCCTTT
TTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCA
GCAGAGCGCAGATACCAAATACTGTTCTTCTAGTGTAGCCGTAGTTAGGCCACCA
CTTCAAGAACTCTGTAGCACCGCCTACATACCTCGCTCTGCTAATCCTGTTACCA
GTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTAT
GAGAAAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACGCCTG
GTATCTTTATAGTCCTGTCGGGTTTCGCCACCTCTGACTTGAGCGTCGATTTTTGT
GATGCTCGTCAGGGGGGCGGAGCCTATGGAAAAACGCCAGCAACGCGGCCTTTT
TACGGTTCCTGGCCTTTTGCTGGCCTTTTGCTCAC
SEQ ID NO: 21 pAAV ITR 130 EF1S synMMUT1 HPRE ITR 141 kana
CCTGCAGGCAGCTGCGCGCTCGCTCGCTCACTGAGGCCGCCCGGGCGTCGGGCG
ACCTTTGGTCGCCCGGCCTCAGTGAGCGAGCGAGCGCGCAGAGAGGGAGTGGCC
AACTCCATCACTAGGGGTTCCTGCGGCCGTAGGTACCGGCTCCGGTGCCCGTCAG
TGGGCAGAGCGCACATCGCCCACAGTCCCCGAGAAGTTGGGGGGAGGGGTCGGC
AATTGAACCGGTGCCTAGAGAAGGTGGCGCGGGGTAAACTGGGAAAGTGATGTC
GTGTACTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAGGTCAG
ATCAGATCTTTGTCGATCCTACCATCCACTCGACACACCCGCCAGCGGCCGCGTT
GGTATCAAGGTTACAAGACAGGTTTAAGGAGACCAATAGAAACTGGGCATGTGG
AGACAGAGAAGACTCTTGGGTTTCTGATAGGCACTGACTCTCTTCCTTTGTCCTGT
TCCCATTTCAGAAGCTTCCGAGCTCTCGAATTCGAGCTCGGTACCTCGCGTGCAT
CTAGATAATCCACCATGCTGAGAGCCAAAAACCAGCTGTTCCTGCTGAGCCCCCA
CTATCTGAGACAGGTCAAAGAAAGTTCCGGGAGTAGACTGATCCAGCAGAGACT
GCTGCACCAGCAGCAGCCACTGCATCCTGAGTGGGCCGCTCTGGCCAAGAAACA
GCTGAAGGGCAAAAACCCAGAAGACCTGATCTGGCACACTCCAGAGGGGATTTC
AATCAAGCCCCTGTACAGCAAAAGGGACACTATGGATCTGCCAGAGGAACTGCC
AGGAGTGAAGCCTTTCACCCGCGGACCTTACCCAACTATGTATACCTTTCGACCC
TGGACAATTCGGCAGTACGCCGGCTTCAGTACTGTGGAGGAATCAAACAAGTTTT
ATAAGGACAACATCAAGGCTGGACAGCAGGGCCTGAGTGTGGCATTCGATCTGG
CCACACATCGCGGCTATGACTCAGATAATCCCAGAGTCAGGGGGGACGTGGGAA
TGGCAGGAGTCGCTATCGACACAGTGGAAGATACTAAGATTCTGTTCGATGGAA
TCCCTCTGGAGAAAATGTCTGTGAGTATGACAATGAACGGCGCTGTCATTCCCGT
GCTGGCAAACTTCATCGTCACTGGCGAGGAACAGGGGGTGCCTAAGGAAAAACT
GACCGGCACAATTCAGAACGACATCCTGAAGGAGTTCATGGTGCGGAATACTTA
CATTTTTCCCCCTGAACCATCCATGAAAATCATTGCCGATATCTTCGAGTACACC
GCTAAGCACATGCCCAAGTTCAACTCAATTAGCATCTCCGGGTATCATATGCAGG
AAGCAGGAGCCGACGCTATTCTGGAGCTGGCTTACACCCTGGCAGATGGCCTGG
AATATTCTCGAACCGGACTGCAGGCAGGCCTGACAATCGACGAGTTCGCTCCTAG
ACTGAGTTTCTTTTGGGGAATTGGCATGAACTTTTACATGGAGATCGCCAAGATG
AGGGCTGGCCGGAGACTGTGGGCACACCTGATCGAGAAGATGTTCCAGCCTAAG
AACTCTAAGAGTCTGCTGCTGCGGGCCCATTGCCAGACATCCGGCTGGTCTCTGA
CTGAACAGGACCCATATAACAATATTGTCAGAACCGCAATCGAGGCAATGGCAG
CCGTGTTCGGAGGAACCCAGAGCCTGCACACAAACTCCTTTGATGAGGCCCTGG
GGCTGCCTACCGTGAAGTCTGCTAGGATTGCACGCAATACACAGATCATTATCCA
GGAGGAATCCGGAATCCCAAAGGTGGCCGATCCCTGGGGAGGCTCTTACATGAT
GGAGTGCCTGACAAACGACGTGTATGATGCTGCACTGAAGCTGATTAATGAAAT
CGAGGAAATGGGGGGAATGGCAAAGGCCGTGGCTGAGGGCATTCCAAAACTGA
GGATCGAGGAATGTGCAGCTAGGCGCCAGGCACGAATTGACTCAGGAAGCGAAG
TGATCGTCGGGGTGAATAAGTACCAGCTGGAGAAAGAAGACGCAGTCGAAGTGC
TGGCCATCGATAACACAAGCGTGCGCAATCGACAGATTGAGAAGCTGAAGAAAA
TCAAAAGCTCCCGCGATCAGGCACTGGCCGAACGATGCCTGGCAGCCCTGACTG
AGTGTGCTGCAAGCGGGGACGGAAACATTCTGGCTCTGGCAGTCGATGCCTCCC
GGGCTAGATGCACTGTGGGGGAAATCACCGACGCCCTGAAGAAAGTCTTCGGAG
AGCACAAGGCCAATGATCGGATGGTGAGCGGCGCTTATAGACAGGAGTTCGGGG
AATCTAAAGAGATTACCAGTGCCATCAAGAGGGTGCACAAGTTCATGGAGAGAG
AAGGGCGACGGCCCAGGCTGCTGGTGGCAAAGATGGGACAGGACGGACATGAT
CGCGGAGCAAAAGTCATTGCCACCGGGTTCGCTGACCTGGGATTTGACGTGGAT
ATCGGCCCTCTGTTCCAGACACCACGAGAGGTCGCACAGCAGGCAGTCGACGCT
GATGTGCACGCAGTCGGAGTGTCCACTCTGGCAGCTGGCCATAAGACCCTGGTGC
CTGAACTGATCAAAGAGCTGAACTCTCTGGGCAGACCAGACATCCTGGTCATGTG
CGGCGGCGTGATCCCACCCCAGGATTACGAATTCCTGTTTGAGGTCGGGGTGAGC
AACGTGTTCGGACCAGGAACCAGGATCCCTAAGGCCGCAGTGCAGGTCCTGGAT
GATATTGAAAAGTGTCTGGAAAAGAAACAGCAGTCAGTGTAAAGACGCGTGGTA
CCTCTAGAGTCGACCCGGGCGGCCTCGAGATAACAGGCCTATTGATTGGAAAGTT
TGTCAACGAATTGTGGGTCTTTTGGGGTTTGCTGCCCCTTTTACGCAATGTGGATA
TCCTGCTTTAATGCCTTTATATGCATGTATACAAGCAAAACAGGCTTTTACTTTCT
CGCCAACTTACAAGGCCTTTCTCAGTAAACAGTATATGACCCTTTACCCCGTTGC
TCGGCAACGGCCTGGTCTGTGCCAAGTGTTTGCTGACGCAACCCCCACTGGTTGG
GGCTTGGCCATAGGCCATCAGCGCATGCGTGGAACCTTTGTGTCTCCTCTGCCGA
TCCATACTGCGGAACTCCTAGCCGCTTGTTTTGCTCGCAGCAGGTCTGGAGCAAA
CCTCATCGGGACCGACAATTCTGTCGTACTCTCCCGCAAGTATACATCGTTTCCAT
GGCTGCTAGGCTGTGCTGCCAACTGGATCCTGCGCGGGACGTCCTTTGTTTACGT
CCCGTCGGCGCTGAATCCCGCGGACGACCCCTCCCGGGGCCGCTTGGGGCTCTAC
CGCCCGCTTCTCCGTCTGCCGTACCGTCCGACCACGGGGCGCACCTCTCTTTACG
CGGACTCCCCGTCTGTGCCTTCTCATCTGCCGGACCGTGTGCACTTCGCTTCACCT
CTGCACGTCGCATGGAGGCCACCGTGAACGCCCACCGGAACCTGCCCAAGGTCT
TGCATAAGAGGACTCTTGGACTTTCAGCAATGTCATCGATATCGTCGACTCGCTG
ATCAGCCTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAAATGA
GGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGA
TGCGGTGGGCTCTATGGCTTCTGAGGCGGAAAGAACCAGCTGGGGCTCGACTAG
ACTAGTCCTGCAGGTACGGCCGCAGGAACCCCTAGTGATGGAGTTGGCCACTCCC
TCTCTGCGCGCTCGCTCGCTCACTGAGGCCGGGCGACCAAAGGTCGCCCGACGCC
CGGGCTTTGCCCGGGCGGCCTCAGTGAGCGAGCGAGCGCGCAGCTGCCTGCAGG
GGCGCCTGATGCGGTATTTTCTCCTTACGCATCTGTGCGGTATTTCACACCGCATA
CGTCAAAGCAACCATAGTACGCGCCCTGTAGCGGCGCATTAAGCGCGGCGGGTG
TGGTGGTTACGCGCAGCGTGACCGCTACACTTGCCAGCGCCTTAGCGCCCGCTCC
TTTCGCTTTCTTCCCTTCCTTTCTCGCCACGTTCGCCGGCTTTCCCCGTCAAGCTCT
AAATCGGGGGCTCCCTTTAGGGTTCCGATTTAGTGCTTTACGGCACCTCGACCCC
AAAAAACTTGATTTGGGTGATGGTTCACGTAGTGGGCCATCGCCCTGATAGACGG
TTTTTCGCCCTTTGACGTTGGAGTCCACGTTCTTTAATAGTGGACTCTTGTTCCAA
ACTGGAACAACACTCAACTCTATCTCGGGCTATTCTTTTGATTTATAAGGGATTTT
GCCGATTTCGGTCTATTGGTTAAAAAATGAGCTGATTTAACAAAAATTTAACGCG
AATTTTAACAAAATATTAACGTTTACAATTTTATGGTGCACTCTCAGTACAATCTG
CTCTGATGCCGCATAGTTAAGCCAGCCCCGACACCCGCCAACACCCGCTGACGCG
CCCTGACGGGCTTGTCTGCTCCCGGCATCCGCTTACAGACAAGCTGTGACCGTCT
CCGGGAGCTGCATGTGTCAGAGGTTTTCACCGTCATCACCGAAACGCGCGAGAC
GAAAGGGCCTCGTGATACGCCTATTTTTATAGGTTAATGTCATGAACAATAAAAC
TGTCTGCTTACATAAACAGTAATACAAGGGGTGTTATGAGCCATATTCAACGGGA
AACGTCGAGGCCGCGATTAAATTCCAACATGGATGCTGATTTATATGGGTATAAA
TGGGCTCGCGATAATGTCGGGCAATCAGGTGCGACAATCTATCGCTTGTATGGGA
AGCCCGATGCGCCAGAGTTGTTTCTGAAACATGGCAAAGGTAGCGTTGCCAATG
ATGTTACAGATGAGACGGTCAGACTAAACTGGCTGACGGAATTTATGCCTCTTCC
GACCATCAAGCATTTTATCCGTACTCCTGATGATGCATGGTTACTCACCACTGCG
ATCCCTGGAAAAACAGCATTCCAGGTATTAGAAGAATATCCTGATTCAGGTGAA
AATATTGTTGATGCGCTGGCAGTGTTCCTGCGCCGGTTGCATTCGATTCCTGTTTG
TAATTGTCCTTTTAACAGCGATCGCGTATTTCGTCTCGCTCAGGCGCAATCACGA
ATGAATAACGGTTTGGTTGATGCGAGTGATTTTGATGACGAGCGTAATGGCTGGC
CTGTTGAACAAGTCTGGAAAGAAATGCATAAACTTTTGCCATTCTCACCGGATTC
AGTCGTCACTCATGGTGATTTCTCACTTGATAACCTTATTTTTGACGAGGGGAAA
TTAATAGGTTGTATTGATGTTGGACGAGTCGGAATCGCAGACCGATACCAGGATC
TTGCCATCCTATGGAACTGCCTCGGTGAGTTTTCTCCTTCATTACAGAAACGGCTT
TTTCAAAAATATGGTATTGATAATCCTGATATGAATAAATTGCAGTTTCATTTGAT
GCTCGATGAGTTTTTCTAATCAGAATTGGTTAATTGGTTGTAACACTGGCAGAGC
ATTACGCTGACTTGACGGGACGGCGCAAGCTCATGACCAAAATCCCTTAACGTG
AGTTTTCGTTCCACTGAGCGTCAGACCCCGTAGAAAAGATCAAAGGATCTTCTTG
AGATCCTTTTTTTCTGCGCGTAATCTGCTGCTTGCAAACAAAAAAACCACCGCTA
CCAGCGGTGGTTTGTTTGCCGGATCAAGAGCTACCAACTCTTTTTCCGAAGGTAA
CTGGCTTCAGCAGAGCGCAGATACCAAATACTGTTCTTCTAGTGTAGCCGTAGTT
AGGCCACCACTTCAAGAACTCTGTAGCACCGCCTACATACCTCGCTCTGCTAATC
CTGTTACCAGTGGCTGCTGCCAGTGGCGATAAGTCGTGTCTTACCGGGTTGGACT
CAAGACGATAGTTACCGGATAAGGCGCAGCGGTCGGGCTGAACGGGGGGTTCGT
GCACACAGCCCAGCTTGGAGCGAACGACCTACACCGAACTGAGATACCTACAGC
GTGAGCTATGAGAAAGCGCCACGCTTCCCGAAGGGAGAAAGGCGGACAGGTATC
CGGTAAGCGGCAGGGTCGGAACAGGAGAGCGCACGAGGGAGCTTCCAGGGGGA
AACGCCTGGTATCTTTATAGTCCTGTCGGGTTTCGCCACCTCTGACTTGAGCGTCG
ATTTTTGTGATGCTCGTCAGGGGGGCGGAGCCTATGGAAAAACGCCAGCAACGC
GGCCTTTTTACGGTTCCTGGCCTTTTGCTGGCCTTTTGCTCAC
SEQ ID NO: 22. AAV ITR 141 base pair
AGGAACCCCTAGTGATGGAGTTGGCCACTCCCTCTCTGCGCGCTCGCTCGCTCAC
TGAGGCCGGGCGACCAAAGGTCGCCCGACGCCCGGGCTTTGCCCGGGCGGCCTC
AGTGAGCGAGCGAGCGCGCAGCTGCCTGCAGG

1. A synthetic methylmalonyl-CoA mutase (MMUT) polynucleotide (synMMUT) selected from the group consisting of:
a) a polynucleotide comprising the nucleic acid sequence of SEQ ID NO: 2; and
b) a polynucleotide having a nucleic acid sequence with at least 85% identity to the nucleic acid sequence of SEQ ID NO:2 and encoding a polypeptide according to SEQ ID NO:3, and having equivalent or increased expression in a host as compared to either expression of SEQ ID NO:2 or SEQ ID NO:1, wherein the polynucleotide does not have the nucleic acid sequence of SEQ ID NO: 1.

2-4. (canceled)

5. The synthetic polynucleotide of claim 1, wherein the synthetic polynucleotide comprises a nucleic acid sequence comprising codons that have been optimized relative to methylmalonyl-CoA mutase (MMUT) polynucleotide sequence (SEQ ID NO: 1).

6. A gene expression cassette comprising the synthetic methylmalonyl-CoA mutase polynucleotide of claim 1.

7. The gene expression cassette of claim 6, further comprising one or more of:
(a) a 5′-inverted terminal repeat sequence (5′-ITR) sequence and a 3′-inverted terminal repeat sequence (3′-ITR) sequence;
(b) a promoter selected from the group consisting of chicken-beta actin promoter, the elongation factor 1 α long promoter (EF1L) (SEQ ID NO:6), the elongation factor 1 α short promoter (SEQ ID NO:7), the long variant of the human alpha 1 antitrypsin promoter (SEQ ID NO: 9) and the short variant of the human alpha 1 antitrypsin promoter (SEQ ID NO: 8);
(c) at least one intron selected from the group consisting of a chimeric intron (SEQ ID NO:12), modified p-globin intron (SEQ ID NO: 13), and a synthetic intron (SEQ ID NO:14);
(d) an apolipoprotein E enhancer selected from the short variant of the apolipoprotein E enhancer (SEQ ID NO: 10) and the long variant of the apolipoprotein E enhancer (SEQ ID NO: 11);
(e) a hepatitis B post transcriptional response element (SEQ ID NO: 15);
(f) a polyadenylation sequence of bovine polyA (SEQ ID NO: 16) or rabbit polyA (SEQ ID NO: 17);
(g) a translation enhancer element; and
(h) a Hepatitis B virus posttranscriptional regulatory element.

8-16. (canceled)

17. A recombinant expression vector comprising the synthetic polynucleotide of claim 1.

18. A recombinant expression vector comprising the gene expression cassette of claim 6.

19. The recombinant expression vector of claim 17, wherein the vector is a recombinant adeno-associated virus (rAAV), said rAAV comprises an AAV capsid, and a vector genome packaged therein, said vector genome comprising:
(a) a 5′-inverted terminal repeat sequence (5′-ITR) sequence;
(b) a promoter sequence;
(c) partial or complete coding sequence for MMUT; and
(d) a 3′-inverted terminal repeat sequence (3′-ITR) sequence.

20. The recombinant expression vector of claim 19, wherein the AAV capsid is from an AAV of serotype 1, 2, 3, 4, 5, 6, 7, 8, 9, 10, 11, 12, 13, rh8, 44.9, rh10, or hu37, and mutants thereof.

21-22. (canceled)

23. The rAAV of claim 19, wherein the promoter is selected from the group consisting of chicken-beta actin promoter, the elongation factor 1 alpha long promoter (EF1L) (SEQ ID NO:6), the elongation factor 1 alpha short promoter (SEQ ID NO:7), the long variant of the human alpha 1 antitrypsin promoter (SEQ ID NO: 9) and the short variant of the human alpha 1 antitrypsin promoter (SEQ ID NO: 8).

24. The rAAV according to claim 19, wherein the rAAV further comprises one or more of:
(a) at least one intron selected from the group consisting of a chimeric intron (SEQ ID NO:12), modified B-globin intron (SEQ ID NO: 13), and a synthetic intron (SEQ ID NO:14);
(b) an apolipoprotein E enhancer selected from the short variant of the apolipoprotein E enhancer (SEQ ID NO: 10) and the long variant of the apolipoprotein E enhancer (SEQ ID NO: 11); and
(c) a hepatitis B post transcriptional response element (SEQ ID NO: 15).

25-32. (canceled)

33. The recombinant expression vector of claim 17, wherein the expression vector comprises the nucleotide sequence of any one of SEQ ID NOs: 18-21.

34-38. (canceled)

39. A composition comprising the synthetic polynucleotide of claim 1 and a pharmaceutically acceptable carrier.

40. A composition comprising the recombinant expression vector of claim 17 and a pharmaceutically acceptable carrier.

41. A method of treating a disease or condition mediated by methylmalonyl-coA mutase, comprising administering to a subject in need thereof a therapeutic amount of a composition comprising the synthetic polynucleotide of claim 1.

42. A method of treating a disease or condition mediated by methylmalonyl-coA mutase, comprising administering to a subject in need thereof a therapeutic amount of a composition comprising the recombinant expression vector of claim 17.

43. A method of treating a disease or condition mediated by methylmalonyl-coA mutase, comprising administering to a subject a composition comprising a methylmalonyl-coA mutase produced using the synthetic polynucleotide of claim 1.

44. The method of claim 41, wherein the disease or condition is Methylmalonic Acidemia (MMA).

45. The method of treating a disease or condition mediated by methylmalonyl-coA mutase of claim 41, comprising administering to a cell isolated from a subject in need thereof a synthetic polynucleotide,
wherein the synthetic polynucleotide is inserted into the cell of the subject via genome editing on the cell of the subject using a nuclease selected from the group of zinc finger nucleases (ZFNs), transcription activator-like effector nucleases (TALENs), the clustered regularly interspaced short palindromic repeats (CRISPER/cas system), and meganuclease re-engineered homing endonucleases on the cell from the subject; and
administering the cell to the subject,
wherein the synthetic polynucleotide is selected from the group consisting of:
(a) a polynucleotide comprising the nucleic acid sequence of SEQ ID NO: 2; and
(b) a polynucleotide having a nucleic acid sequence with at least 85% identity to the nucleic acid sequence of SEQ ID NO:2 and encoding a polypeptide according to SEQ ID NO:3, and having equivalent or increased expression in a host as compared to either expression of SEQ ID NO:2 or SEQ ID NO:1, wherein the polynucleotide does not have the nucleic acid sequence of SEQ ID NO:1.

46. A method of treating a disease or condition mediated by methylmalonyl-coA mutase, comprising administering to a subject a composition comprising a methylmalonyl-coA mutase produced using the recombinant expression vector of claim 17.

47-50. (canceled)

51. A method for monitoring treatment of a disease or condition mediated by methylmalonyl-coA mutase, wherein a therapeutic amount of a composition comprising the recombinant expression vector of claim 17 has been administered to a subject in need thereof, comprising detection of expression of synMMUT1 using RNA in situ hybridization with a synMMUT1 nucleic acid probe.


SEQUENCE LISTING (22 sequences)

SEQ ID NO: 1 - DNA, 2253 bp, Homo sapiens
atgttaagagctaagaatcagctttttttactttcacctcattacctgaggcaggtaaaagaatcatcaggctccaggctcatacagcaacgacttctacaccagcaacagccccttcacccagaatgggctgccctggctaaaaagcagctgaaaggcaaaaacccagaagacctaatatggcacaccccggaagggatctctataaaacccttgtattccaagagagatactatggacttacctgaagaacttccaggagtgaagccattcacacgtggaccatatcctaccatgtatacctttaggccctggaccatccgccagtatgctggttttagtactgtggaagaaagcaataagttctataaggacaacattaaggctggtcagcagggattatcagttgcctttgatctggcgacacatcgtggctatgattcagacaaccctcgagttcgtggtgatgttggaatggctggagttgctattgacactgtggaagataccaaaattctttttgatggaattcctttagaaaaaatgtcagtttccatgactatgaatggagcagttattccagttcttgcaaattttatagtaactggagaagaacaaggtgtacctaaagagaagcttactggtaccatccaaaatgatatactaaaggaatttatggttcgaaatacatacatttttcctccagaaccatccatgaaaattattgctgacatatttgaatatacagcaaagcacatgccaaaatttaattcaatttcaattagtggataccatatgcaggaagcaggggctgatgccattctggagctggcctatactttagcagatggattggagtactctagaactggactccaggctggcctgacaattgatgaatttgcaccaaggttgtctttcttctggggaattggaatgaatttctatatggaaatagcaaagatgagagctggtagaagactctgggctcacttaatagagaaaatgtttcagcctaaaaactcaaaatctcttcttctaagagcacactgtcagacatctggatggtcacttactgagcaggatccctacaataatattgtccgtactgcaatagaagcaatggcagcagtatttggagggactcagtctttgcacacaaattcttttgatgaagctttgggtttgccaactgtgaaaagtgctcgaattgccaggaacacacaaatcatcattcaagaagaatctgggattcccaaagtggctgatccttggggaggttcttacatgatggaatgtctcacaaatgatgtttatgatgctgctttaaagctcattaatgaaattgaagaaatgggtggaatggccaaagctgtagctgagggaatacctaaacttcgaattgaagaatgtgctgcccgaagacaagctagaatagattctggttctgaagtaattgttggagtaaataagtaccagttggaaaaagaagacgctgtagaagttctggcaattgataatacttcagtgcgaaacaggcagattgaaaaacttaagaagatcaaatccagcagggatcaagctttggctgaacgttgtcttgctgcactaaccgaatgtgctgctagcggagatggaaatatcctggctcttgcagtggatgcatctcgggcaagatgtacagtgggagaaatcacagatgccctgaaaaaggtatttggtgaacataaagcgaatgatcgaatggtgagtggagcatatcgccaggaatttggagaaagtaaagagataacatctgctatcaagagggttcataaattcatggaacgtgaaggtcgcagacctcgtcttcttgtagcaaaaatgggacaagatggccatgacagaggagcaaaagttattgctacaggatttgctgatcttggttttgatgtggacataggccctcttttccagactcctcgtgaagtggcccagcaggctgtggatgcggatgtgcatgctgtgggcataagcaccctcgctgctggtcataaaaccctagttcctgaactcatcaaagaacttaactcccttggacggccagatattcttgtcatgtgtggaggggtgataccacctcaggattatgaatttctgtttgaagttggtgtttccaatgtatttggtcctgggactcgaattccaaaggctgccgttcaggtgcttgatgatattgagaagtgtttggaaaagaagcagcaatctgtataa

SEQ ID NO: 2 - DNA, 2253 bp, Artificial Sequence
  synthetic
atgctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaa

SEQ ID NO: 3 - protein, 750 aa, Homo sapiens [sequence omitted]

SEQ ID NO: 4 - DNA, 130 bp, adeno-associated virus 2
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 5 - DNA, 145 bp, adeno-associated virus 2
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 6 - DNA, 1179 bp, Homo sapiens
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 7 - DNA, 232 bp, Homo sapiens
cggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 8 - DNA, 251 bp, Homo sapiens
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtg

SEQ ID NO: 9 - DNA, 392 bp, Homo sapiens
tgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 10 - DNA, 189 bp, Homo sapiens
cctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagag

SEQ ID NO: 11 - DNA, 318 bp, Homo sapiens
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgaga

SEQ ID NO: 12 - DNA, 133 bp, Homo sapiens
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 13 - DNA, 447 bp, Homo sapiens
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaaggaattc

SEQ ID NO: 14 - DNA, 165 bp, Artificial Sequence
  synthetic
gtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcag

SEQ ID NO: 15 - DNA, 726 bp, Homo sapiens
ataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatc

SEQ ID NO: 16 - DNA, 127 bp, Bos taurus
gatctttttccctctgccaaaaattatggggacatcatgaagccccttgagcatctgacttctggctaataaaggaaatttattttcattgcaatagtgtgttggaattttttgtgtctctcactcg

SEQ ID NO: 17 - DNA, 127 bp, Oryctolagus cuniculus; identical to SEQ ID NO: 16

SEQ ID NO: 18 - DNA, 7302 bp, Artificial Sequence
  synthetic
cttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattcttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtagccatgctctagcgatcgcggtacctctagaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgcgtccaccatgctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgtctagaactagtcctgcaggtagagcatggctacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctg

SEQ ID NO: 19 - DNA, 6134 bp, Artificial Sequence
  synthetic
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtcctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcgcgccaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgacgcgtccaccatgctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattag

SEQ ID NO: 20 - DNA, 6486 bp, Artificial Sequence
  synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgtaggtaccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattcgagctcggtacctcgcgtgcatctagataatccaccatgctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaaagacgcgtggtacctctagagtcgacccgggcggcctcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagacggtcagactaaactggctgacggaatttatgcctcttccgaccatcaagcattttatccgtactcctgatgatgcatggttactcaccactgcgatccctggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcac

SEQ ID NO: 21 - DNA, 6497 bp, Artificial Sequence
  synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgtaggtaccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattcgagctcggtacctcgcgtgcatctagataatccaccatgctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaaagacgcgtggtacctctagagtcgacccgggcggcctcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagacggtcagactaaactggctgacggaatttatgcctcttccgaccatcaagcattttatccgtactcctgatgatgcatggttactcaccactgcgatccctggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcac

SEQ ID NO: 22 - DNA, 141 bp, adeno-associated virus 2
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg